(19) United States
(12) Patent Application Publication
(10) Pub. No.: US 2010/0081130 A1
(43) Pub. Date: Apr. 1, 2010

(54) Multifunctional particles providing cellular uptake and magnetic motor effect

(76) Inventors: Jin-Kyu Lee, Seoul (KR); Myung-Haing Cho, Seoul (KR); Seung-Bum Park, Seoul (KR); Tae-Jong Yoon, Seoul (KR)

Correspondence Address: Customer No. 70416;Perkins Coie LLP, Patent - LA, P.O. Box 1208, Seattle, WA, 98111-1208, US

(21) Appl. No.: 12/030,848

(22) Filed: Feb. 13, 2008

Related U.S. Application Data
(62) Division of application No. 11/220,969, filed on Sep. 8, 2005, now abandoned.

(51) Int. Cl. C12Q 1/68; C12Q 1/02

(52) U.S. Cl. 435/6; 435/29

(57) ABSTRACT

Preparation of novel multifunctional particles and nanomaterials having a useful combination of magnetic and optical properties and biocompatibility. The internalization efficiencies in various in vitro cell studies have been investigated, and the external magnetic motor effect on the floating cells internalized with magnetic nanoparticles were clearly observed, for the first time. The particle surfaces can be derivatized with, for example, DNA or antibodies. The system is stable, versatile, and well-controlled. Novel gene delivery can be achieved using nanoparticles as a carrier.

This application is a divisional of and incorporates by reference U.S. patent application Ser. No. 11/220,969 filed Sep. 8, 2005.

### BACKGROUND

The present patent application generally relates to magnetic particles and cells. The application is divided into three parts, and each of the parts relate to magnetic particles and cells. Background for Part One is first provided, followed by additional background for Parts Two and Three.

Magnetic materials are important for commercial biological applications including, for example, diagnostics and biosensors. See, for example, US Patent publications 2005/0130167 to Josephson et al.; 2003/0092029 to Josephson et al.; 2005/0025971 to Cho et al.; 2004/0208825 to Carpenter et al.; 2004/0109824 to Hinds et al.; and U.S. Pat. Nos. 6,514,481 to Prasad et al and 6,767,635 to Bahr et al. In many cases, it is important to mediate and precisely control the interface of an inorganic phase of the material with an organic phase of the material. The organic phase may provide properties such as biomolecular recognition or biocompatibility, whereas the inorganic phase may provide a useful property such as the magnetism. Magnetic materials can be in particulate form, wherein the particles can be generally spherical in shape. Alternatively, they can be non-spherical in shape showing an aspect ratio. The particles can have dimensions extending down to the nanometer scale, e.g., 100 nm or less, and magnetic nanoparticles (MNP) can be commercially exploited because their small size allows them to penetrate into cells.

In particular, magnetic ferrite nanoparticles (sometimes called ferrofluids) are one important type of magnetic material. They have been widely used in various applications such as smart seal magnetic circuits (reference 1), audio speakers (reference 2), and magnetic domain detectors (reference 3). Recently, magnetic nanoparticles have also been suggested for new applications in high-density magnetic data storage (reference 4), magnetic resonance imaging (reference 5), catalyst supporters (reference 6), and biomedical applications such as magnetic carriers for bio-separation (reference 7) and enzyme and protein immobilization (reference 8) and contrast-enhancing media (reference 9). In addition, nanoparticles have been coated with a shell of stable and biocompatible material such as silica (SiO₂) to avoid potential toxic effects on cells (see references 10-12).

Imaging cells is important. For example, transmission electron microscopy (TEM) or magnetic resonance imaging (MRI) has been used to observe magnetic nanoparticles incorporated within cells. However, TEM and MRI are not convenient for in situ monitoring. Hence, a sensitive and easy technique for monitoring the nanoparticles in cells in situ is desirable. Confocal laser scanning microscopy (CLSM) is a highly sensitive detection technique specific to the fluorescence wavelength of the dye used.

In addition, the present patent application also reports surface charge effects for magnetic particles (see Part Two below) and additional background is now provided for Part Two.

Reports in the literature have demonstrated new biological applications of various nanomaterials of metal, metal oxide, semiconductor, and carbon (C₆₀ and carbon nanotube, CNT) for imaging and diagnostic materials, delivery carriers, and so on (reference 17). On the other hand, the possible harmfulness of nanomaterials has also been pointed out and preliminary toxicity data have been reported (reference 18). Proper surface coating with biocompatible materials can be important in order to prevent the possible emergence of metal toxicities (reference 19) and to introduce surface groups which can be functionalized for the purpose of bioconjugation. The synthesis of biocompatible nanomaterials has thus come to be an intensively studied field by many researchers, therefore, and size-controllable and multifunctional core-shell nanoparticles have attracted much attention. Among various shell coating materials, silica (SiO₂) is a very promising candidate having good biocompatibility and chemical stability in organisms (reference 20). Silica-coated core-shell nanomaterials have been recently synthesized through various methods for biological application, and organic fluorescent dyes have been incorporated into a silica shell for more extensive applications (references 21, 22). U. Wiesner et. al. have reported that organic fluorescent dye embedded in silica nanobeads showed long-term fluorescent stability and significantly reduced photobleaching phenomena (reference 23).

In addition, the present patent application also reports surface functionalization (see Part Three below) and additional background is now provided for Part Three.

Magnetic nanoparticles (MNP) have been used in various areas such as in the manufacture of bearings, seals, lubricants, heat carriers, and in printing, recording, and polishing media (reference 33). One of the rapidly developing research subjects involving MNP is its application in the biological system, including its application in magnetic resonance imaging (MRI), targeted drug delivery, rapid biological separation, bio-sensor, and therapy (reference 34). The exploration of the interaction between nanostructured materials and living systems is of fundamental and practical interest, and it opens new doors to novel commercial applications which are interdisciplinary in nature, including “NanoBio science”. MNPs exhibited potential for in vitro and in vivo biomedical application (reference 35), and the biodistribution of MNP is strongly influenced by its size, charge, and surface chemistry (reference 36). Recently published reports indicate that magnetic nano (or micro) particles (Fe₃O₄) conjugated with various target molecules or antibodies can be used to target specific cells in vitro (reference 37). However, the non-covalent surface modification of nanoparticles has a serious limitation for biological applications because the exposed metal ion on the surface of nanoparticles can cause metal elemental toxicities in cells (in vivo model) (reference 38). Silica (SiO₂) modification can be used because it is a good biocompatible material and resistant to decomposition in vivo (reference 39). Hence, silica-coated core-shell nanoparticles have been studied over the past decade (reference 40) and these were recently synthesized with a functionalized surface for bioconjugation by various methods for application (reference 41) in biological systems.

In sum, a need exists to provide better use of magnetic particles for cellular study and manipulation, including better control of particle structure and improved versatility, stability, and biocompatability. In many cases in the prior art, one or more of the required features for a complex application is missing, and it can be difficult to achieve the combination of properties needed for a particular application. A need exists for a single system which is versatile and can be modified to meet different challenges that different applications present. In particular, commercial issues such as reproducibility, cost, and scale-up ability are often lacking in the prior art.

### SUMMARY

Described herein is a well-controlled, versatile magnetic particle system which can be quantitatively analyzed and provides numerous advantages for commercial applications. In one embodiment, a magnetic motor effect has been developed wherein magnetic particles disposed inside of cells can be used to move cells in a magnetic field. Gene delivery and specific targeting are also described and experimentally demonstrated.

In particular, one embodiment provides a magnetic cell composition comprising a cell comprising a plurality of magnetic particles inside the cell, wherein the number of magnetic particles is sufficiently high to provide the cell with magnetic movement when the cell is exposed to an external magnetic field but not sufficiently high to cause cytotoxicity. The cell can be, for example, a eukaryotic cell or a human, animal, or plant cell. The particles can have an average particle size of about 100 nm or less, or more particularly, about 30 nm to about 80 nm. The particles can comprise a magnetic core and a non-magnetic shell. The particles can further comprise a fluorescent dye for confocal laser scanning microscopy. The particles can comprise a magnetic core and a surrounding shell of inorganic glass which comprises covalently bound fluorescent dye for confocal laser scanning microscopy and which further comprises a covalently bound surface agent which enhances the cellular uptake for the particles. In one important embodiment, the particles are free of components which provide specific recognition. The number of particles in the cell can be about 10⁴ or more, or more particularly, about 10⁵ or more.

The cell movement can be carried out with the cell in, for example, a Petri dish and a magnetic strength applied of about 0.3 Tesla on the outside of the Petri dish. The cell can move at a speed of at least about 0.5 mm per second, and even at a speed of at least about 1 mm per second.

Another embodiment provides a composition comprising: a plurality of particles having an average particle size of about 100 nm or less, wherein the particles comprise: (i) a core comprising magnetic material, and (ii) a glassy inorganic oxide shell disposed around the core which is covalently bound to at least one luminescent organic dye which is distributed through the glass inorganic oxide shell, wherein the shell further comprises a surface agent which is covalently bound to the shell and enhances the particle cellular uptake. In one embodiment, the particles are free of components which provide a specific recognition. The agent to enhance cellular uptake can achieve cellular uptake non-specifically. The shell can comprise an inorganic oxide material such as, for example, silica or alumina, and in particular silica. The core can also be a particle comprising an organic polymer stabilizer.

Another important embodiment provides that the particle surface is adapted to include surface ionic charges or surface functional groups which can react to further derivatize the particles. For example, the surfaces of the particles can be derivatized with DNA, genes, antibodies, and proteins.

One embodiment provides a composition comprising: a plurality of particles having an average particle size of about 100 nm or less, wherein the particles comprise: a core comprising magnetic material, and a glassy inorganic oxide shell disposed around the core which is covalently bound to at least one luminescent organic dye which is distributed through the glassy inorganic oxide shell, wherein the shell further comprises a surface agent which is covalently bound to the shell and provides the surface with an anionic or cationic charge.

Another embodiment is a composition comprising: a plurality of particles having an average particle size of about 100 nm or less, wherein the particles comprise: a core comprising magnetic material, and a glassy inorganic oxide shell disposed around the core which is covalently bound to at least one luminescent organic dye which is distributed through the glassy inorganic oxide shell, wherein the shell further comprises a surface agent which is covalently bound to the shell and provides the surface with an amine functional group.

Embodiments described herein include both individual particles and collections of particles, as well as material compositions comprising these particles.

For example, also provided is a specific embodiment for a nanoparticle comprising: (i) a metal-containing magnetic core comprising a polyvinylpyrrolidone stabilizer, (ii) a silica shell formed in the presence of the stabilizer around the core, wherein the silica shell comprises (1) a luminescent dye homogeneously distributed through the shell and does not photobleach, and (2) a surface comprising ethylene oxide repeat units, and wherein the size of the particle is about 30 nm to about 80 nm as measured by TEM.

Other embodiments described herein include methods of making and methods of using nanoparticles and nanoparticulates compositions, as well as methods of making and using cells comprising the magnetic nanoparticles. Particularly important applications include magnetic separation, specific targeting, and gene delivery. In particular, gene delivery with nanoparticle carrier is believed to be an important, novel feature.

The advantages for one or more of the embodiments described herein are numerous and include, for example, (1) reproducibility, (2) ability to do large scale synthesis, (3) good long term stability for the core particle as well as the core-shell particle, (4) control over the amount of dye in the particle, (5) precise control over shell thickness, including silica shell thickness, (6) ability to quantitatively design and control the synthesis of the particle, (7) control over particle size including ability to generate small particles sizes such as 100 nm or less, (8) minimal or no cytotoxicity, (9) magnetic movement including fast movement, (9) ability to quantitatively introduce target marker, (10) lack of photobleaching, (11) large number of charge sites on particle surface, and (12) multi-functionality can be monitored simultaneously by fluorescence microscope and magnetic resonance imaging for in vitro or in vivo model. In general, the system is very well controlled and susceptible to quantitative analysis.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. TEM images of Co ferrite@silica (core-shell) magnetic nanoparticles with controlled shell thicknesses. (A) TEOS/MNP=0.12 mg/4 mg, scale bar: 100 nm, (B) TEOS/MNP=0.06 mg/4 mg, scale bar: 50 nm and (C) TEOS/MNP=0.03 mg/4 mg, scale bar: 50 nm. As the ratio of TEOS/MNP (w/w) was increased, the shell thickness was continuously increased, and this trend can be directly applied to the synthesis of dye-labeled and surface-modified core-shell magnetic nanoparticles, MNP@SiO₂(RITC or FITC) and MNP@SiO₂(RITC or FITC)-PEG.

FIG. 2. CLSM images of breast cancer cells (MCF-7) after 24 h of growth in media containing PEG-modified MNP@SiO₂(RITC)-PEG (A-C) or unmodified MNP@SiO₂(RITC) nanoparticles (D-F). (A and D: fluorescence images, B and E: bright field images, C and F: overlay of the fluorescence and bright field images) From the difference in fluorescence intensity of these images, it was corroborated that the PEG groups on the surface enhanced the internalization efficiency of nanoparticles.

FIG. 3. CLSM z-sectioned images of breast cancer cells (MCF-7) obtained by using MNP@SiO₂(RITC)-PEG and MNP@SiO₂(FITC)-PEG (A and D: top slices, B and E: middle slices, C and F: bottom slices). The white arrows indicated the position of nucleus.

FIG. 4. After MCF-7 cells were attached onto a glass cover slip, the culture solution mixed with MNP@SiO₂(RITC)-PEG solution was loaded. Cells were investigated for 30 min with a moving picture video camera and up to 48 h by consecutively taking pictures. (A) Several representative pictures were captured for every 5 min period. As time elapsed, one could clearly determine the location of the nucleus (white arrow) owing to the uptake into the cytoplasm. (B) After saturation of internalization, the media was removed and carefully washed with new culture solution. Then, CLSM measurements of cells on the washed glass cover slip were conducted again. (left: fluorescence image, middle: bright field image, and right: merged image)

FIG. 5. Schematic illustration of the overall synthetic procedure of MNP@SiO₂(RITC)-PEG and the movement of cells internalized with magnetic nanoparticles by an external magnetic force.

FIG. 6. Optical microscope images of floating B tumor cells. Images were captured every 0.2 seconds from a moving picture. External magnetic field direction is toward upper-left corner and B tumor cells that had sunk to the bottom moved relatively slowly to that direction (white circles), while floating B tumor cells moved much faster (blue and red arrows). The black arrows denote the standard direction of cell movements at the bottom surface of the dish.

FIG. 7. Schematic illustration of PEG modified organic dye-labeled magnetic nanoparticles from bare nanoparticles.

FIG. 8. TEM images and ED patterns of bare Co ferrite nanoparticles (left) and MNP@SiO₂(RITC) (right), Scale bar=100 nm.

FIG. 9. Magnetic properties of core-shell nanoparticles having different silica shell thickness, measured by Vibrating Sample Magnetometer (VSM, Lake Shore Model 7304); (a) TEOS/MNP=0.12 mg/4 mg, scale bar: 100 nm, (b) TEOS/MNP=0.06 mg/4 mg, scale bar: 50 nm and (c) TEOS/MNP=0.03 mg/4 mg, scale bar: 50 nm, as described in FIG. 1 of the main text. The saturation magnetization (Mₛ) of the core-shell nanoparticle was decreased as the shell thickness was increased. It was mainly attributed to the volume of the non-magnetic silica coating layer to the total sample volume. The coercivity (H_{c}) values were exactly same for all the samples, on the contrary, corroborating that the core materials are same as bare Co ferrite magnetic nanoparticle.

FIG. 10. TEM image of MNP@SiO₂(RITC)-PEG (scale bar=100 nm).

FIG. 11. TEM image of silica-coated Co ferrite (core-shell) magnetic nanoparticles, MNP@SiO₂(RITC). The average size is 58 nm. In an inset image at higher magnification, the core part of Co ferrite nanoparticle is clearly observed as a darker seed than silica shell owing to the higher electron density.

FIG. 12. Surface charges of modified MNP@SiO₂(RITC). The maximum potential peaks of MNP@SiO₂(RITC)-PMP, MNP@SiO₂(RITC), MNP@SiO₂(RITC)-PEG, and MNP@SiO₂(RITC)-PTMA were measured at −50, −16.7, 2.4, and 35.7 mV, respectively. The effective zeta-potentials in aqueous solution were measured by particle characterizer “ZetaSizer-Nano” (Sysmex corp., Kobe, Japan), and the values were averaged from 5 times assay data.

FIG. 13. CLSM images of A549 cells incorporated with various modified core-shell nanoparticles at the same concentration. Incorporation efficiencies of the nanoparticles decrease in the order of MNP@SiO₂(RITC)-PEG (A), MNP@SiO₂(RITC)-PTMA (B)≈MNP@SiO₂(RITC) (C), MNP@SiO₂(RITC)-PMP (D), based on the relative fluorescent intensities from RITC. The black dot in each cell shows the position of nucleus due to the impermeability of nanoparticles into nucleus membrane.

FIG. 14. Optimization of the hybrid ratio of MNP@SiO₂(RITC)-PTMA with plasmid DNA. (A) electrophoretic gel shift bands of A. DNA utilized as a size maker (lane 1), pcDNA3.1/CT-GFP with various ratio of MNP@SiO₂(RITC)-PTMA (lanes 2˜5); (B) electrophoretic gel shift bands of pcDNA3.1/CT-GFP itself (lane 1), with neutral MNP@SiO₂(RITC) (lanes 2 and 3), λ DNA maker (lane 4), and with positive MNP@SiO₂(RITC)-PTMA (lane 5 and 6).

FIG. 15. CLSM and TEM micrograph images of transfected A549 cells by pcDNA3.1/CT-GFP delivered by MNP@SiO₂(RITC)-PTMA vesicle. Green fluorescence image (A), bright field image (B), orange fluorescence image (C), and merged image (D). Green fluorescence from GFP expression was observed in most of cells and localized orange fluorescence from localized MNP@SiO₂(RITC)-PTMA was also detected in each cells. Cross-sectioned TEM micrograph images (E) with enlarged images where MNP@SiO₂(RITC) nanoparticles are localized.

FIG. 16. Schematic illustration of the overall synthetic procedure preparing core-shell nanoparticle, MNP@SiO₂(RITC), and surface modifying with various Si-compounds. The red spots in the silica shell are depicting the incorporation of APS-RITC.

FIG. 17. Dynamic light scattering (DLS) data of the MNP@SiO₂(RITC) core-shell nanoparticle in H₂O, measured at different concentrations and calculated from the light scattering theorem. The averaged dynamic size of core-shell nanoparticles is determined as 58.1 nm.

FIG. 18. IR Characterization of modified MNP@SiO₂(RITC) core-shell nanoparticles with various organosilicon compounds

FIG. 19. MTT assay data of (A) MNP@SiO₂(RITC); (B) MNP@SiO₂(RITC)-PEG; (C) MNP@SiO₂(RITC)-PMP; and (D): MNP@SiO₂(RITC)-PTMA for MCF-7 cell (black), A549 (light gray), and NL20 (gray), respectively, with different concentrations.

FIG. 20. Cross-sectioned TEM micrograph images of MNP@SiO₂(RITC)-PTMA/DNA complexes internalized into the A549 cells. B and D (or C and E) images are enlarged from red dot square (or blue dot square) in panel A.

FIG. 21. TEM image (A) of MNP@SiO₂(FITC), high magnitude image of core-shell nanoparticles (upper inset, a portion of the Co ferrite nanoparticle is darker than that of silica shell on account of the electron density), and ED-pattern shows a crystallinity (bottom inset, see the XRD data in supporting information). Their average size is approximately ˜60 nm. T₂ weighted negative contrast MR images of MNP@SiO₂(FITC) in aqueous solution (C) and the contrast difference correlated with the concentration (concentration of B left: 0.2 mg·mL⁻¹ and B right: 2.0×10⁻⁴ mg·mL⁻¹).

FIG. 22. CLSM images of SP2/0 leukemia cells and A549 lung cancer cells on treatment with MNP@SiO₂(FITC)-Ab_{CD-10} after 24 h of growth in media. In the case of A549 lung cancer cell (D˜F), the fluorescence was not detected because the CD-10 antibody modified nanoparticles could be specifically bound to receptors on SP2/0 cell surface (A˜C). (A and D: green fluorescence images, B and E: bright field images, C and F: show the merged fluorescence and bright field images.

FIG. 23. Optical microscope images of the targeted SP2/0 floating cells (white dots) moving due to the application of an external magnetic field. Before (A) and after (B) the application of a magnetic field by external magnet (˜0.3 T); the red dotted circle indicates location of the magnet. The floating cells move fast in the direction of the magnet (supporting information). The green back ground color is not due to fluorescence but due to the back light from the optical microscope equipment.

FIG. 24. CLSM images of nuclear DAPI (blue) stained MCF-7 cells after targeting and internalization of the MNP@SiO₂(FITC)-Ab_{HER-2} and MNP@SiO₂(RITC). Green fluorescence image (A), bright field image (B), red fluorescence image (C), and the merged image (D). A white arrow in C is indicated the nucleus.

FIG. 25. TEM micrographs of cell containing with MNP@SiO₂(RITC) (A) and targeted cell by HER-2 modified nanoparticles (B). In A, subcellular organelles, for example, lysosome and mitocondria, were observed with MNP@SiO₂(RITC)s (white arrows) nearby. Antibody-modified nanoparticles (red arrows) were clearly targeted on the outer cell membrane.

FIG. 26. Schematic illustration of the steps involved in antibody modified MNP@SiO₂(FITC) synthesis. A: CoFe₂O₄ magnetic nanoparticle, B: MNP@SiO₂(FITC) core-shell nanoparticle, C: MNP@SiO₂(FITC)-PEG/NH₂ dual-fabricated core-shell nanoparticle, D: Maleimide terminated smart silica core-shell magnetic nanoparticle, E: MNP@SiO₂(FITC)-Ab multifunctional nanoparticle for specific binding.

FIG. 27. Powder X-ray Diffraction pattern of MNP@SiO₂(FITC)

FIG. 28. Dynamic light scattering (DLS) data of the MNP@SiO₂(FITC) magnetic core-silica shell nanoparticle in H₂O. The correlation graph and size distribution graph are not shown, and below graph is calculated from the light scattering theorem. The size of core-shell nanoparticles is exactly determined 58.1 nm.

FIG. 29. Vibrato Sample Magnetometer (VSM) data of MNP@SiO₂(FITC) magnetic core-silica shell nanoparticles.

FIG. 30. MTT assay data of MNP@SiO₂(FITC) for MCF-7 cell (black), A549 (light gray), and NL20 (gray). In this assay, the cell viability was maintained at greater than 85% in all groups indicating that the core-shell nanoparticle do not show acute cytotoxicity to various cells at the level of a few tens of micrograms (80 μg mL⁻¹) within 48 h.

FIG. 31. TEM images of MNP@SiO₂(RITC) uptakes cell. B is enlarged from black dot square in A. the core-shell structure of nanoparticles were clearly shown at the cytoplasm of the cell.

### DETAILED DESCRIPTION

### Introduction

Merely for sake of organization, the specification is divided into three parts: (1) Part One describes, for example, particle synthesis, cellular uptake, and magnetic movement of cells; (2) Part Two describes, for example, ionic functionalization of the particle surface and an associated surface charge effect; and (3) Part Three describes, for example, non-ionic functionalization of the particle surface including amino and maleimide functionalization.

The following paper provides description for various embodiments and is incorporated by reference in its entirety including the figures, supplementary information, experimental section, and references: Yoon et al., “Multifunctional Nanoparticles Possessing a ‘Magnetic Motor Effect’ for Drug or Gene Delivery,” Angew. Chem. Int. Ed. 2005, 44, 1068-1071.

Additional references provide background knowledge and skill which can be used in the practice of the various embodiments described herein. For example, magnetic materials, including particles and nanoparticles, are described in, for example, Poole and Owens, Introduction to Nanotechnology, Wiley, 2003, Chapter 7, “Nanostructured Ferromagnetism,” pages 165-193, which is incorporated by reference in its entirety. In particular, ferrofluids are described on pages 186-192. Chapter 4 describes properties of individual particles including magnetism (pages 72-102). The total magnetic moment of the electron can comprise both spin and orbital magnetic moments, and fundamentals of magnetism for metals such as iron, manganese, and cobalt are described. Ferromagnetism, paramagnetism, ferrimagnetism, antiferromagnetism, and superparamagnetism are described. Magnetic domains and grain are described, as well as particles and powders. Hard and soft ferromagnetism are described. See also, for example, Awschalom and von Molnar, “Physical Properties of Nanometer-scale Magnets,” in Nanotechnology, G. Timp. Ed., 1999, Chapter 12.

In addition, preparation and applications of magnetic particles are generally reviewed in (1) Willner and Katz, Angew. Chem. Int. Ed., 2004, 43, 6042-6108, including section 7 beginning at page 6078, (2) Pankhurst et al., J. Phys. D.: Appl. Phys. 36 (2003) R167-R181, (3) Tartaj et al., J. Phys. D.: Appl. Phys. 36 (2003) R182-R197, and (4) Berry et al., J. Phys. D.: Appl. Phys. 36 (2003) R198-R206, which are hereby incorporated by reference in their entirety.

In addition, biological and pharmaceutical applications of magnetic particles are described in, for example, US Patent publications 2005/0130167 to Josephson et al.; 2003/0092029 to Josephson et al.; 2005/0025971 to Cho et al.; 2004/0208825 to Carpenter et al.; 2004/0109824 to Hinds et al.; and U.S. Pat. Nos. 6,514,481 to Prasad et al and 6,767,635 to Bahr et al.

### Part One

### Cell Composition

Magnetic cell compositions are provided comprising a cell which further comprises a plurality of magnetic particles inside the cell. The number of magnetic particles is sufficiently high to provide the cell with magnetic movement when the cell is exposed to an external magnet, but not sufficiently high to cause cytotoxicity.

An advantage is that the magnetic particles can be inside the cell rather than merely on the surface of the cell. This allows, for example, greater flexibility in designing the particle to not have specific recognition agents for specific types of cells. This also allows higher numbers of particles to become associated with the cell. Hence, for example, smaller particles like nanoparticles can be used.

The type of cell is not particularly limited but in general can be governed by the particular application. Cell lines and cultured cells are known in the art. Cells can be prepared and examined either in vitro or in vivo. Both healthy and unhealthy cells can be used. Diseased cells can be used. Cancerous or tumor cells can be used. The cells can be prokaryotic or eukaryotic. The cells can be human cells, animal cells, mammalian cells, epithelial cells, endothelial cells, organ cells, nerve cells, muscle cells, blood cells, somatic cells, sex cells, root cells, skin cells, glioma cells, plant cells, specialized cells, yeast cells, algae cells, or bacterial cells. Stem cells can be used. The cells can be used in a natural state or after genetic engineering. Mixtures of cells can be used. One cell organisms can be used. Cells can be alive or dead, although generally living cells are more important.

The shape of the cell is not particularly limited. It can be like cubes, coils, boxes, snowflakes, corkscrews, rods, saucers, rectangles, tiny balls, or even blobs of jelly. The size of the cell is not particularly limited. Cell size or dimension can be, for example, about 10 microns to about 100 microns.

Cell monolayers can be used.

Cells are desired which can float in liquids and be moved by a magnetic field as described herein.

The magnetic particles can be applied to and taken up by individual cells, collections of cells, or tissues.

### Magnetic Particles and Size

There are no particular limits to the preparation method for the magnetic particles. Wet, dry, or vacuum methods can be used. Preparation methods include, for example, grinding bulk materials, precipitation from solution, coprecipitation, microemulsions, polyols, high temperature decomposition of organic precursors, solution techniques, aerosol/vapor methods, spray pyrolysis, plasma atomization, and laser pyrolysis. Capping ligands can be used to prevent aggregation.

Magnetic particles can be microspheres, nanospheres, or ferrofluids. They can obey Coulomb's law and can be manipulated by an external magnetic field.

There are no particular limitations to the type of magnetic material in the magnetic particle. Magnetic metals can be used including one or more of the following metals: iron, cobalt, zinc, cadmium, nickel, gadolinium, chromium, copper, manganese, and their oxides. Alloys can be used including metal alloys of gold, silver, platinum, and copper. The magnetic material can also be a free metal ion, a metal oxide, a chelate, or an insoluble metal compound. Examples include Fe₃O₄, Fe2O₃, FeₓPt_{y}, COₓPt_{y}, MnFeₓO_{y}, CoFeₓO_{y}, NiFeₓO_{y}, CuFeₓO_{y}, ZnFeₓO_{y}, and CdFeₓO_{y} wherein x and y can vary depending on the method of synthesis. Metal coatings can be used including gold, silver, iron, cobalt, zinc, cadmium, nickel, gadolinium, chromium, copper, and manganese, and an alloy thereof. The particle can comprise terbium or europium. The magnetic particle can be a monocrystalline iron oxide nanoparticle (MION), a chelate of gadolinium, or a superparamagnetic iron oxide (SPIO). Ferrites can be used. Commercial iron oxide nanoparticles, which can be used in commerce as contrast agents, of maghemite (Endorem® and Resovit®) can be used.

Whether they are nanometer or micrometer scale, a collection or plurality of magnetic particles can have narrow polydispersity or broad polydispersity and can be characterized by average particle size or diameter. Known methods such as TEM can be used to measure particle size. In general, monodisperse materials can be used which have substantial uniformity in size and shape.

The plurality of particles can have an average total particle size, encompassing both core and shell, of about 200 nm or less, or more particularly, of about 100 nm or less, or more particularly, about 1 nm to about 100 nm, or more particularly, about 10 nm to about 100 nm, or more particularly, about 25 nm to about 90 nm, or more particularly, about 30 nm to about 80 nm, or more particularly, about 40 nm to about 70 nm. The average total particle size can be sub-micron.

### Number of Magnetic Particles Inside the Cell

The number of magnetic particles inside the cell can be a saturation number. In other words, the cell can take up particles to a certain limit at which point substantially no more particles are taken up. The number of particles is sufficiently high to provide the cell with magnetic movement when the cell is exposed to an external magnetic field. However, the number should not be so high that cytotoxicity results. The working examples below and references cited herein describe how to measure the number of particles inside the cell, movement in an external magnetic field, and cytotoxicity and cell death.

The number of magnetic particles can be, for example, at least about 10⁴ or more, or at least about 10⁵ or more, or at least about 10⁶ or more, or at least about 10⁷ or more, or at least about 10⁸ or more, or at least about 10⁹ or more.

One can also measure the number of magnetic particles on the outside surface of the cell membrane but these are not inside the cell.

### Particle Core

The plurality of particles can have a core-shell structure which can be characterized by the average size or diameter of the particle core which can be, for example less than about 50 nm, or less than about 25 nm, or about 5 nm to about 20 nm. The core-shell particles can have a magnetic core and a non-magnetic shell. The core can be substantially or entirely made of inorganic material. The core can be homogeneous or heterogeneous. The core can be crystalline. Mixtures of materials can be used in the core. For example, one component in the core may provide magnetic properties, whereas another component may provide surface adsorption properties.

The core should also be stable, including stable in water. For example, cationic surface sites on the core surface can improve stability and prevent aggregation. Some aggregation of the core can be allowed. For example, five to 30 core particle can aggregate before further treatment. Aggregation can be measured by comparison of DLS particle size with TEM particle size. In many cases, it is desirable that particle size by TEM generally equals the particle size by DLS. A larger DLS value can indicate stability problems. For core particles prepared by coprecipitation, surface treatment with extra Fe(NO₃)₃ can generate a thin layer of Fe(OH)³ structure on the surface; this can be followed with treatment by 1 N HNO₃ for generating cationic surface sites.

The core can also include on its surface a stabilizer to prevent aggregation of the particles during synthesis of the core, sometimes also called in the arts a coating, an organic linker, or a capping ligand, and there are no particular limitations on the selection of the stabilizer. The stabilizer can improve colloidal stability. It can also improve the control over the shell synthesis by, for example, adjusting solubility or dispersability. The stabilizer can mediate the formation of a glassy shell such as for example silica formation. Although the present embodiments are not limited by theory, some interaction may be present between the stabilizer and the glass material such as silica. General types of stabilizers include hydrophobic monolayers, positively or negatively charged hydrophilic monolayers, and polymer layers. The stabilizer can be, for example, a synthetic organic polymer such as a vinyl polymer, a neutral polymer, a polyelectrolyte, or a water soluble polymer. An example is poly(vinyl pyrrolidone) (PVP). The molecular weight of the polymer is not particularly limited as long as stabilization can be achieved. Phospholipids coatings can be used. Organosilane stabilizers can be used. Mixtures of stabilizers can be used. In general, biocompatible materials such as dextran, polyvinylalcohol (PVA), or phospholipids are of interest.

The stabilizer can be adsorbed or covalently bound to the core particle.

The stabilizer can be a surface coating on the core but generally is associated with the core rather than the shell because generally the core is synthesized with use of the stabilizer.

The particle core also should not generate toxicity generally in case the shell is removed.

### Particle Shell

The plurality of particles having a core-shell structure can be characterized by the average size or thickness of the particle shell which can be, for example determined by measuring the total size of the particle, and the size of the core, and determining the difference between the two measurements.

The shell can comprise an inorganic glass which comprises covalently bound fluorescent dye useful for, for example, confocal laser scanning microscopy. The dye can be homogeneously distributed throughout the shell which can help prevent photobleaching when the shell is a rigid material. In most cases, photostability is desired. Photobleaching can harm attempts to quantitatively monitor the system.

The shell can further comprise a covalently bound surface agent which enhanced cellular uptake. For example, ethyleneoxy units can be introduced onto the surface of the shell to help with cellular uptake and biocompatibility.

The inorganic glass can be an oxide material including an inorganic oxide material. It can be amorphous or crystalline. Materials capable of sol-gel chemistry can be used. Alumina and silica can be used. In particular, silica is an important material for the shell. Titania can be formed but can be toxic in biological applications. The glass should be hard at 25° C., and any glass transition temperature should be well above room temperature. Mixtures of materials can be used in the shell. The shell is not particularly limited by the degree of porosity.

In general, shell materials are preferred which do not degrade or are removed over time in a cell. The shell should not generate toxicity.

### Shell Dye Component

The shell can comprise a luminescent or fluorescent dye useful, for example, for fluorescent microscopy and/or confocal laser scanning microscopy. The dye can be functionalized so that it can be covalently linked to the glassy shell. The particular dye used is not particularly limited. Mixtures of dyes can be used. Emission wavelengths of, for example, about 450 nm to about 650 nm, or about 500 nm to about 600 nm can be used.

The dye, when introduced into the shell, should allow for homogeneous distribution in the shell. Moreover, it should not provide photobleaching. Distribution in the shell can reduce or eliminate toxicity and photoinstability effects.

### Specific Recognition

A basic and novel feature is that in many embodiments the particles can be free of components which provide specific recognition. If specific recognition is desired, known recognition, specific-binding systems in biochemistry can be used. These agents for specific recognition can be disposed on the particle surface.

### Methods of Making Particles and Cells

There are no particular limits on how the particles and cells are made. However, the core particle is preferably made by coprecipitation using a polymer stabilizer such as PVP which provides a well-controlled core for further well-controlled synthesis of the shell.

One embodiment is a method of making a magnetic cell composition comprising a cell comprising a plurality of magnetic particles inside the cell, wherein the number of magnetic particles is sufficiently high to provide the cell with magnetic movement when the cell is exposed to an external magnetic field but not sufficiently high to cause cytotoxicity. The cell can be provided. The magnetic particles can be synthesized. The cell can be exposed to the magnetic particles so they are taken up inside the cell. For example, exposure can be carried out until the cell uptake is saturated. The number of particles in the cell can be monitored until the desired number is achieved for a particular application.

Another embodiment is a method of making a composition comprising: a plurality of particles having an average particle size of about 100 nm or less, wherein the particles comprise: (1) a core comprising magnetic material, and (2) a glassy inorganic oxide shell disposed around the core which is covalently bound to at least one luminescent organic dye which is distributed through the glassy inorganic oxide shell, wherein the shell further comprises a surface agent which is covalently bound to the shell and enhances the particle cellular uptake. The core can be synthesized. The core can be modified with a polymer stabilizer which facilitates formation of the shell. The glassy inorganic shell can be synthesized around the core which results in substantial homogeneous distribution throughout the shell of the dye. The shell can be further treated so that it comprises a surface agent which is covalently bound to the shell and enhances the particle cellular uptake.

### Cellular Uptake

There are no particular limits on how the particles are taken up by the cell. In one embodiment, the cell nucleus does not take up the magnetic particles. Endocytosis can be used including receptor mediated energy dependent endocytosis.

### Magnetic Cell Properties

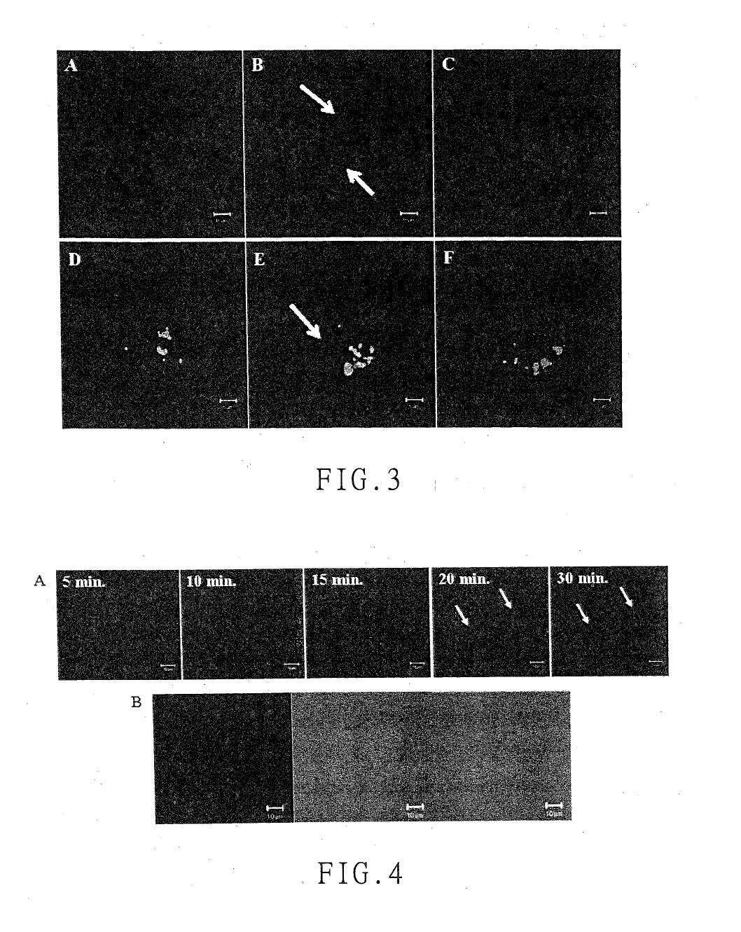

To measure the magnetic cell properties, methods similar to those used in magnetic separation studies can be used. See, for example, Pankhurst et al., “Applications of Magnetic Nanoparticles in Biomedicine,” J. Phys. D: Appl. Phys., 36 (2003) R167-R181, section 3 on magnetic separation including FIG. 3. These methods typically include tagging the cell with the magnetic material, and separating the tagged cell from other non-tagged cells or other materials.

For example, the magnetic movement can be tested with use of normal Petri dish or test tube and magnets and magnetic field components known in the art. For example, the movement can be carried out with the cell in a Petri dish and a magnetic strength applied of about 0.3 Tesla on the outside of the Petri dish. No carrier medium is needed to move the cell whether by flowing or floating. Rather, movement is carried out by movement of the magnetic field. Cells can be stationary and induced to move, or can be moving as a result of non-magnetic forces and induced to change direction or speed of movement by the magnetic force.

The cells can be moved at relatively high speed solely based on the magnetic movement force. Speeds can reach, for example, at least about 0.5 mm per second, or at least about 1 mm per second.

Cells can be floating in aqueous media for the speed measurements.

Rapid throughput designs can be used for commercial processes.

### Applications

Many related applications exist including both relatively more basic studies which are also important background and foundation for relatively more applied studies in commercial applications. Applications can be either in vivo or in vitro. Applications can be therapeutic or diagnostic. Examples include drug or gene delivery, transfection, diagnostics, sensors, bioseparation, cellular uptake studies, cell sorting, hyperthermia agents, and other applications noted in the background section and references cited herein. Other examples include magnetic targeting of drugs, genes, and radiopharmaceuticals, magnetic resonance imaging, contrast agents, diagnostics, immunoassays, RNA and DNA purification, gene cloning, and cell separation and purification.

Additional applications include, for example, study of the toxicity of the nanoparticle for in vivo applications, magnetic patch effect, and real-time monitoring of the nanoparticle circulation process in the living body. These basic investigative results on core-shell magnetic nanoparticles can yield commercially valuable applications in cell separation, biological labeling and detection, drug and gene delivery carriers, diagnostics, and the like.

### Part One

### Working Examples

For Part One, non-limiting working examples are further provided.

Incorporation of a fluorescent dye into the silica shell was carried out. The polyvinylpyrrolidone (PVP) method has been widely applied to particles having ionic surface charges to generate sol-gel silica coating with variable thickness by varying the amount of tetraethoxysilane (TEOS) loaded (reference 13). This example describes the preparation, employing a modified PVP method, of cobalt ferrite magnetic nanoparticles coated with a shell of amorphous silica, which contain luminescent organic dyes such as rhodamine B isothiocyante (RITC, orange color, λ_{max.(em.)}=555 nm) or fluorescein isothiocyanate (FITC, green color, λ_{max.(em.)}=518 nm) on the inside of the silica shell and biocompatible poly(ethylene glycol) (PEG) on the outside. It was of interest to determine whether the fluorescence characteristics of the organic dye could be utilized in comparing by CLSM the efficiency of uptake into cells of the magnetic nanoparticles with and without PEG modification. Monitoring the movement of doped cells under an external magnetic field was also important because of its use in bio-separation and related applications.

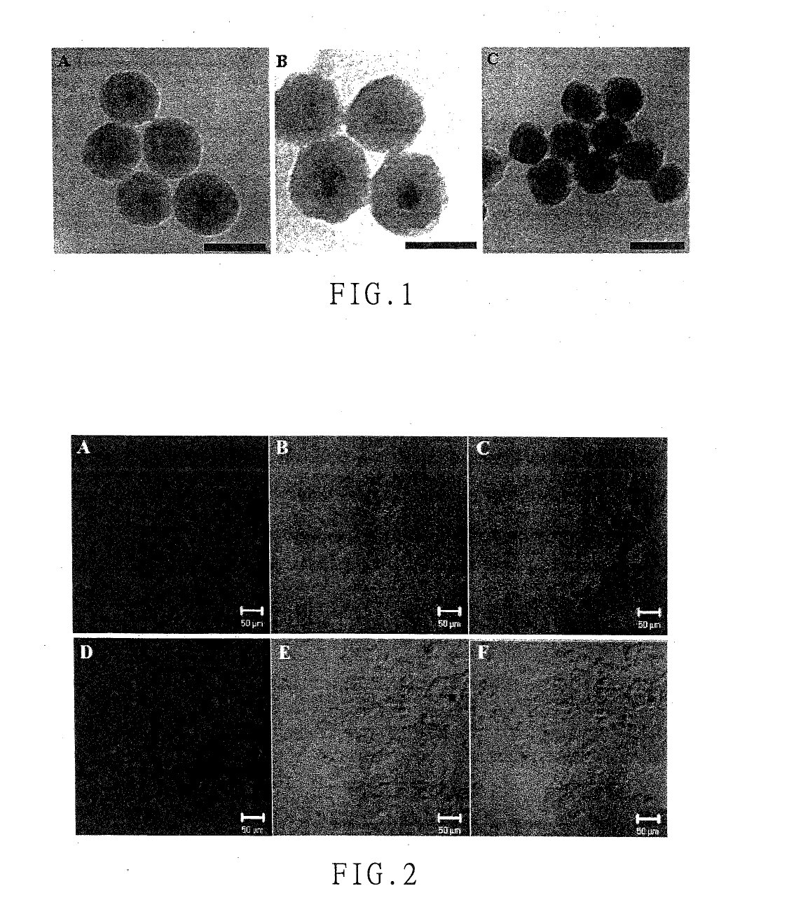

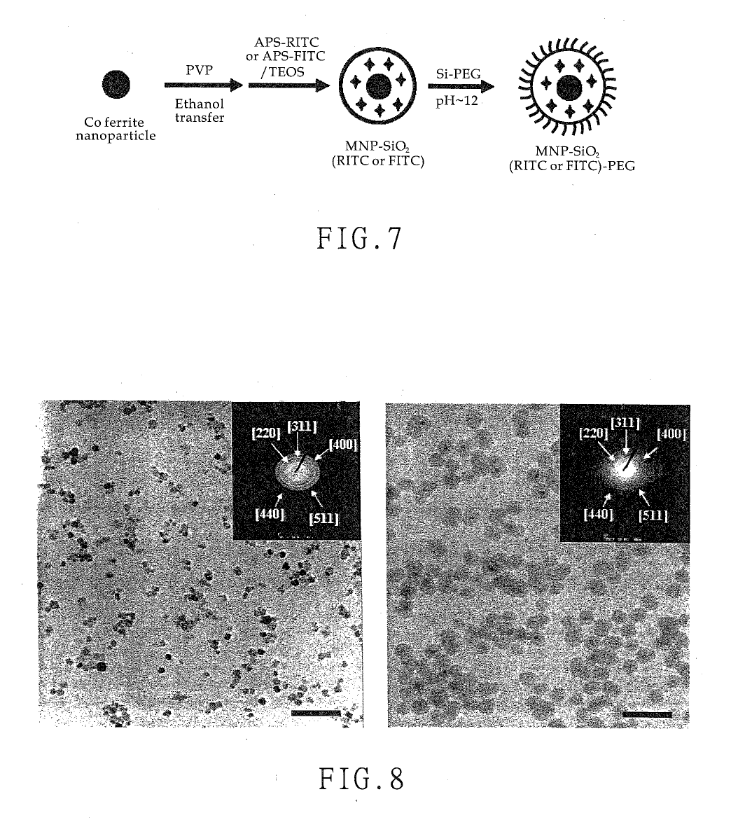

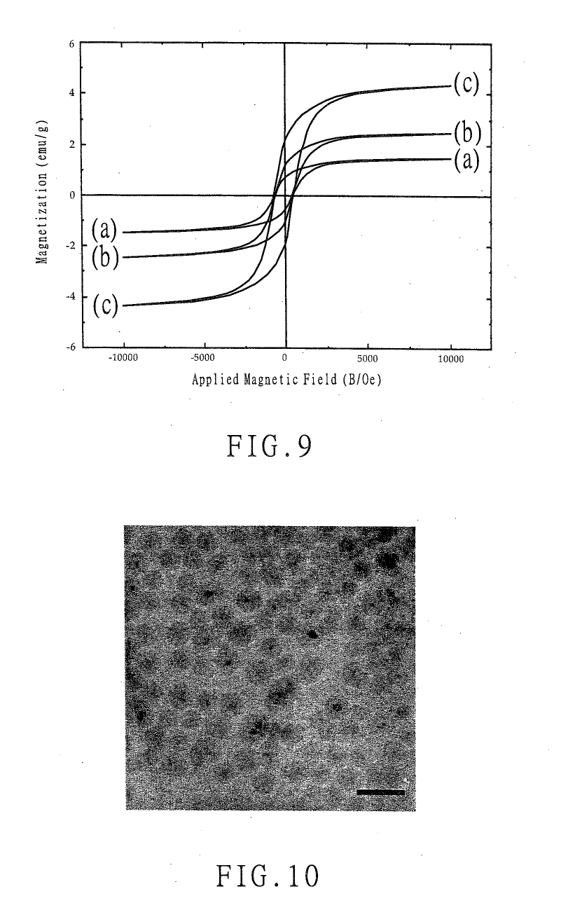

Water-soluble bare cobalt ferrite magnetic nanoparticles of average size about 9 nm, synthesized by a slight modification of a known co-precipitation method from FeCl₃.6H₂O and CoCl₂.6H₂O in hot basic NaOH solution (reference 14), were stabilized with PVP to make them homogeneously dispersed in ethanol. When the mixed solution of TEOS and dye-modified silane compound, synthesized from 3-aminopropyltriethoxysilane (APS) and dye-isothiocyanate (reference 15), was polymerized on the surface of PVP-stabilized cobalt ferrites by adding ammonia solution as a catalyst to form organic-dye incorporating cobalt ferrite-silica core-shell nanoparticles, the ratio of the concentrations of cobalt ferrite magnetic nanoparticles (Co ferrite MNP) and TEOS had been carefully selected to prevent the homogeneous nucleation of silica and to control the shell thickness of core-shell ferrite-silica magnetic nanoparticles. The TEM images in FIG. 1 of silica-coated Co ferrite nanoparticles prepared using different ratios of TEOS/MNP show that the thickness of the silica shell can be precisely controlled to produce core-shell nanoparticles with diameters ranging from 30 nm to 80 nm as the amount of TEOS per 4 mg MNP increased from 0.03 to 0.12 mg. The crystallinity and magnetic properties of the core material did not change upon coating with silica (see FIGS. 8 and 9). PEG was attached to the silica shell surface by adding PEG-Si(OMe)₃ solution after the shell formation was complete (see FIG. 7). This trend could be directly applied to the synthesis of dye-labeled and surface-modified core-shell magnetic nanoparticles, MNP@SiO₂(RITC or FITC) and MNP@SiO₂(RITC or FITC)-PEG. Attachment of PEG did not significantly change the size of the nanoparticles as determined by TEM measurements (FIG. 10). The average diameter of the organic dye-labeled Co ferrite@silica (core-shell) magnetic nanoparticles used in this study was about 50 nm as shown in FIG. 1B.

Although photobleaching can be a common problem when fluorescent dyes are used, no significant photobleaching was observed in this rigid matrix system, as reported for another system (reference 16), and thus fluorescence intensity could be used to quantitatively analyze the quantity of core-shell nanoparticles incorporated into cells. The CLSM images in FIG. 2 show breast cancer cells (MCF-7) after 24 h of growth in media containing MNP@SiO₂(RITC)-PEG (PEG-modified nanoparticle, FIG. 2A-2C) and MNP@SiO₂(RITC) (unmodified, FIG. 2D-2F). From the overlay images (FIGS. 2C and 2F) of the fluorescence images (FIGS. 2A and 2D) and bright field images (FIGS. 2B and 2E), respectively for both samples, it seems that the surface modification by PEG enhances the incorporation of Co ferrite MNP into cells. Thus all subsequent experiments were conducted using MNP@SiO₂(RITC)-PEG.

If dyes having different fluorescence emission wavelengths are used, the position of the nanoparticles containing different dyes could be selectively monitored simultaneously at the respective emission wavelength. The CLSM images in FIG. 3 show breast cancer cells (MCF-7) internalized with MNP@SiO₂(RITC)-PEG or MNP@SiO₂(FITC)-PEG, clearly emitting different wavelengths, namely orange light from RITC and green light from FITC. By changing the focus distances, furthermore, CLSM could slice the cell images at different z positions to clearly show that emission did not emerge from the organic dye-labeled core-shell magnetic nanoparticles adsorbed on the cell membrane surface, but from what was delivered into the cytoplasm of living cells. It was also confirmed that core-shell magnetic nanoparticles could not penetrate the nucleus, showing no emission from the nucleus as depicted in the series of sliced images in FIGS. 3B and 3E.

For the time-dependent studies of the uptake process of MNP@SiO₂(RITC)-PEG nanoparticles by live MCF-7 cells, MCF-7 cells were attached onto a glass cover slip and the culture solution containing MNP@SiO₂(RITC)-PEG nanoparticles was loaded and fluorescence images were taken every 5 min in consecutive real-time CLSM investigation (FIG. 4). As time elapsed, the dark region of the cytoplasm area in the cell faded away and turned into an orange emissive region owing to the uptake of MNP@SiO₂(RITC)-PEG, and the position of the nucleus became clearly visible as marked with white arrows (FIG. 4A). The internalization of a dye labeled core-shell nanoparticle into the cell seemed to be saturated within 30 min, and no significant intensity difference in the cytoplasm area nor emission from nucleus region were detected during the next 48 hours. After the saturation of internalization, the culture solution containing an excess amount of MNP@SiO₂(RITC)-PEG nanoparticles was removed and carefully washed with new culture solution, followed by CLSM measurement again to ensure that the fluorescent emissions came from the internalized MNP@SiO₂(RITC)-PEG nanoparticles (FIG. 4B); all the emission from the culture solution containing MNP@SiO₂(RITC)-PEG nanoparticles around the cells was removed. The internalization process seems to follow the mechanism of normal endocytosis, and it occurs as a general phenomenon of the internalization of core-shell magnetic nanoparicles into various cells including mammalian lung normal cells (NL-20), lung cancer cells (A-549) and breast cancer cells (MCF-7). After 24 h. starvation, MCF-7, NL-20 and A-549 cells were treated with MNP@SiO₂(RITC or FITC)-PEG and the cell viability was measured by MTT assay. In this condition, the cell viability was maintained at greater than 90% in all groups corroborating the fact that the Co ferrite@silica core-shell magnetic nanoparticle, MNP@SiO₂(RITC or FITC)-PEG, did not show acute cytotoxicity to various cells at the level of a few tens of micrograms (80 μg/ml) within 48 h.

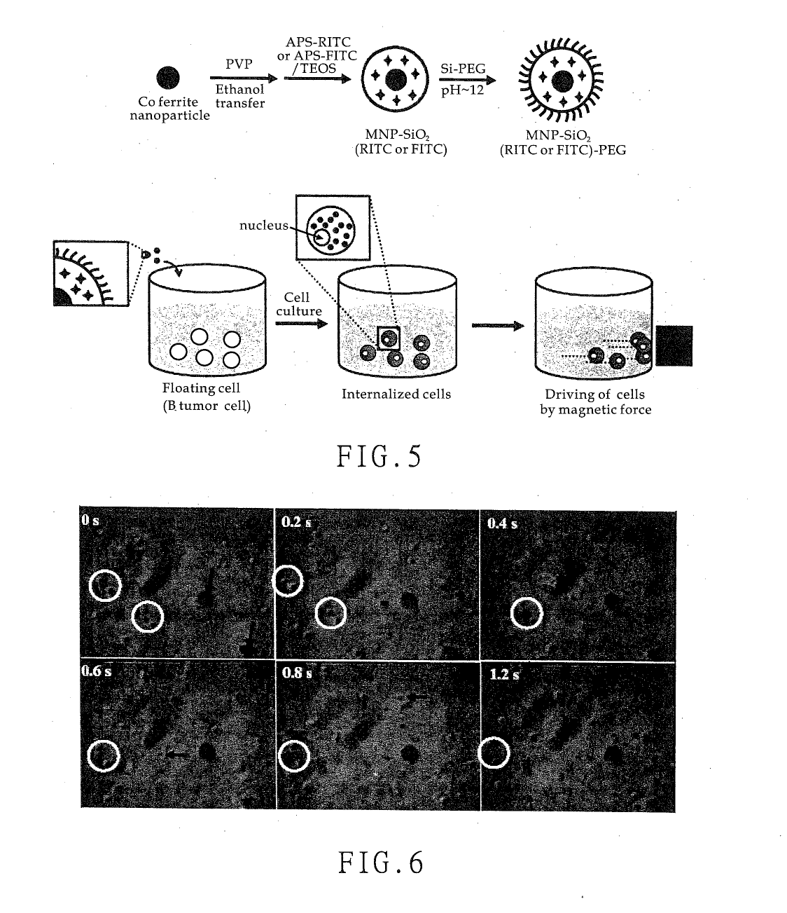

Once it was shown by fluorescent experiments and ICP-AES measurements that a considerable quantity of the magnetic nanoparticles was incorporated into cells (uptake about 10⁵ nanoparticles per cell), monitoring was carried out for the movement of cells internalized with magnetic nanoparticles under an external magnetic field, which can be a major advantage of magnetic nanoparicles for bioapplications such as cell separation, and drug or gene delivery carrier (FIG. 5).

Microscope images in FIG. 6 were captured every 0.2 second from the moving picture focused in the area near the container wall (within about 1 cm distance) while an external magnetic field was applied with a commercial Nd—Fe—B magnet (˜0.3 Tesla) on the outside of the petri-dish (upper-left position in FIG. 6), containing floating B tumor cells internalized with MNP@SiO₂(RITC)-PEG nanoparticles. As was clearly observed in the captured microscope images of FIG. 5, B tumor cells that had sunk (marked with white circles) to the bottom of the petri-dish moved relatively slowly at a speed of about 0.2 mm/sec probably due to the interaction with the bottom surface. However, floating cells not sunk on the bottom (marked with red and blue arrows) moved very fast at a speed of 1.0 mm/sec. When the external magnet was removed and reapplied from the outside of petri-dish, the movement of cells was halted and restarted again. Moving the position of external magnet could also change the direction of the cell movement.

This was a clear observation of a “magnetic motor effect”: cell movement as a result of applying an external magnetic field to internalized cells with magnetic nanoparticles. Surprisingly, the speed of the cell movement was quite fast with the external magnetic force generated by applying a commercial permanent magnet, confirming that the amount of internalized nanomagnets in our system was sufficiently high to show a magnetic motor effect while not causing any cytotoxicity.

### Experimental Section

Chemicals. FeCl₃.6H₂O, CoCl₂.6H₂O, and Fe(NO₃)₃.9H₂O were purchased from Sigma-Aldrich (St. Louis, Mo.). RITC and FITC organic dyes were from Fluka (Switzerland). Silicon compounds such as APS, TEOS and PEG-Si(OMe)₃ were from Gelest (Morrisville, Pa., USA). These chemicals were used without further purification.

Preparation of MNP@SiO₂(RITC or FITC)-PEG. 34.7 mL of cobalt ferrite solution (20 mg MNP/mL solution in water) was added to 0.65 mL of polyvinylpyrrolidone solution (PVP; M_{W} 55,000 Da, 25.6 g/L in H₂O), and the mixture was stirred for 1 day at room temperature. The PVP-stabilized cobalt ferrite nanoparticles were separated by addition of aqueous acetone (H₂O/acetone=1/10, v/v) and centrifugation at 4000 rpm for 10 min. The supernatant was removed and the precipitated particles were redispersed in 10 ml ethanol. Multi gram scale preparation of PVP-stabilized cobalt ferrite nanoparticles was reproduced in this modified synthetic method. Trimethoxysilane modified by rhodamine β isothiocyante (RITC) was prepared from 3-aminopropyltriethoxysilane (APS) and rhodamine β isothiocyante under nitrogen using a standard Schlenk line technique (references 10, 15). A mixed solution of TEOS and RITC-modified trimethoxysilane (TEOS/RITC-silane molar ratio=0.3/0.04) was injected into the ethanol solution of PVP-stabilized cobalt ferrite. Polymerization was initiated by adding 0.86 mL of ammonia solution (30 wt % by NH₃) as a catalyst produced cobalt ferrite-silica core-shell nanoparticles containing organic-dye. These nanoparticles were dispersed in ethanol and precipitated by ultra-centrifugation (18,000 rpm, 30 min). The purified core-shell nanoparticles (45 mg) were redispersed in 10 ml absolute ethanol and then treated with 125 mg (0.02 mmol) of 2-[methoxy(polyethyleneoxy)propyl]trimethoxysilane (PEG-Si(OMe)₃), CH₃O(CH₂CH₂O)_{6˜9}CH₂CH₂CH₂Si(OCH₃)₃, at pH about 11 (adjusted with NH₄OH). The resulting MNP@SiO₂(RITC)-PEG was washed and centrifuged in EtOH several times and characterized by IR spectroscopy to show the clear increment of C—H stretching band at 2800-2900 cm⁻¹. MNP@SiO₂(FITC)-PEG could also be prepared by a similar method except for the usage of FITC-silane instead of RITC-silane. All the prepared multifunctional magnetic nanoparticles were characterized by TEM, FT-IR, VSM, UV-Vis. absorption and emission spectroscopy, confirming the coexistence of each feature.

Cell culture. Breast cancer cells (mammary gland adeno-carcinoma, MCF-7), normal human bronchial epithelial cells (NL-20), and lung cancer cells (A-549) were ordered from American Type Culture Collection (ATCC, Manassas, Va.). MCF-7 cells were grown in DMEM (Cambrex Bio Science, Walkersville, Md.) containing 10% FBS (v/v) and 40 μl of MNP@SiO₂(RITC or FITC)-PEG (2 mg/ml). NL-20 and A-549 were grown in RPMI (Cambrex Bio Science) under the same conditions. All cells were cultured in Lab-Tek glass chamber slide (Nalge Nunc International, Naperville, Ind.) in order to observe fluorescence emission by confocal laser scanning microscopy (CLSM).

MTT assay. The cells were incubated in a 96-well plate. At the end of the incubation period, 50 μl of MTT (3(4,5-dimethylthiazol-2-yl) 2,5-diphenyltetrazolium bromide; Sigma-Aldrich) in PBS (0.2 mg/ml) was added to each well (final concentration of 0.4 mg/ml) and cultures were incubated in 5% CO₂ for 4 h at 37° C. Then the culture medium was carefully removed by pipetting and formazan crystals were dissolved in 150 μl DMSO. After 10 min agitation on a shaker, absorbance was measured at 490 nm and 620 nm for test and reference solutions, respectively.

Confocal laser scanning microscopy (CLSM). 3D image reconstructions of organic dye-labeled nanoparticles were obtained with a Zeiss LSM 510 CLSM equipped with a computer-controlled scan stage. An argon laser for RITC excitation at 543 nm (488 nm for FITC) was used for imaging. For each cell, more than 10 optical planes were scanned by changing the focal length to detect the nanoparticles at different locations within the cell. In experiments involving live cells, an exclusive culture chamber was used to maintain a cell culture temperature of 37° C.

Determination of the quantity of nanoparticles in cells. The total number of cells in the magnetic motor experiment (FIG. 5) was estimated to be about 4.0×10⁵ by using a hemacytometer chamber. ICP-AES measurement, after the cells containing nanoparticles were destroyed and nanoparticles were completely dissolved with concentrated HCl, reveals the quantity of Co ion in each cell to be about 10⁻¹³ mmol. From calculations, each 9 nm CoFe₂O₄ nanoparticle contains about 10⁻¹⁸ mmol of Co ions. Therefore, the number of magnetic nanoparticles in each cell in our magnetic motor effect experiments can be estimated to be of the order of about 10⁵.

In summary, the organic dye-labeled Co ferrite@silica (core-shell) magnetic nanoparticles have been prepared by a modified polyvinylpyrolidone (PVP) method and sol-gel process. The thickness of the silica shell could be controlled by adjusting the ratio of magnetic nanoparticle (MNP)/tetraethoxysilane (TEOS) and dye-modified silane. Core-shell magnetic nanoparticles could also be labeled with two different organic dyes such as rhodamine β isothiocyante (RITC) and fluorescent isothiocyanate (FITC), and the nanoparticle surface could be modified with bio-inert poly(ethylene glycol) (PEG) groups, providing unique multifunctional magnetic and optical properties along with biocompatibility. Also investigated was the internalization efficiencies of MNP@SiO₂(RITC or FITC), and MNP@SiO₂(RITC or FITC)-PEG in various in vitro cell studies. One clearly observed the external magnetic motor effect on the floating cells internalized with magnetic nanoparticles.

### REFERENCES FOR PART ONE

1. K. Furumura, H. Sugi, Y. Murakami, H. Asai, 1986, U.S. Pat. No. 4,598,914.
2. J. A. King, 1977, U.S. Pat. No. 4,017,694.
3. a) D. H. Han, H. L. Luo, Z. Yang, J. Magn. Magn. Mater. 1996, 161, 376-378; b) H. Fujiwara, J. Appl. Phys. 1993, 73, 5757-5762.
4. a) Q. Song, Z. J. Zhang, J. Am. Chem. Soc. 2004, 126, 6164-6168; b) H. Zeng, J. Li, J. P. Liu, Z. L. Wang, S. Sun, Nature 2002, 420, 395-398; c) C. Liu, B. Zou, A. J. Rondinone, Z. J. Zhang, J. Am. Chem. Soc. 2000, 122, 6263-6267.
5. a) J. M. Perez, L. Josephson, T. O'Loughlin, D. Högemann, R. Weissleder, Nat. Biotechnol. 2002, 20, 816-820; b) J. M. Perez, T. O'Loughlin, F. J. Simeone, R. Weissleber, L. Josephson, J. Am. Chem. Soc. 2002, 124, 2856-2857; c) L. Babes, B. Denizot, G. Tanguy, J. J. L. Jeune, P. Jallet, J. Colloid Interf. Sci. 1999, 212, 474-482.
6. T. J. Yoon, W. Lee, Y. S. Oh, J. K. Lee, New J. Chem. 2003, 227-229.
7. a) P. S. Doyle, J. Bibette, A. Bancaud, J. Viovy, Science 2002, 295, 2237; b) H. Gu, P. Ho, K. W. T. Tsang, L. Wang, B. Xu, J. Am. Chem. Soc. 2003, 125, 15702-15703; c) D. Wang, J. He, N. Rosenzweig, Z. Rosenzweig, Nano Lett. 2004, 4, 409-413.
8. a) D. Cao, P. He, N. Hu, Analyst 2003, 128, 1268-1274; b) T. Mirzabekov, H. Kontos, M. Farzan, W. Marasco, J. Sodroski, Nat. Biotechnol. 2000, 18, 649-654; c) J. P. Chen, W. S. Lin, Enzyme Microb. Tech. 2003, 32, 801-811; d) K. Nishimura, M. Hasegawa, Y. Ogura, T. Nishi, K. Kataoka, H. Handa, M. Abe, J. Appl. Phys. 2002, 91, 8555-8556; e) I. Willner, E. Katz, Angew. Chem. Int. Ed. 2003, 42, 4576-4588.
9. a) E. X. Wu, H. Tang, K. K. Wong, J. Wang, J. Magn. Reson. Imaging 2004, 19, 50-58; b) J. M. Perez, L. Josephson, R. Weissleder, ChemBioChem. 2004, 5, 261-264.
10. Y. Lu, Y. Yin, B. T. Mayers, Y. Xia, Nano Lett. 2002, 2, 183-186.
11. M. A. Correa-Duarte, M. Giersig, N. A. Kotov, L. M. Liz-Marzán, Langmuir 1998, 14, 6430-6435.
12. a) A. Kros, M. Gerritsen, V. S. I. Sprakel, N. A. J. M. Sommerdijk, J. A. Jansen, R. J. M. Nolte, Sensor Actuat. B-Chem. 2001, 81, 68-75; b) Y. Shchipunov, J. Colloid Interf. Sci. 2003, 268, 68-76.
13. a) C. Graf, D. L. J. Vossen, A. Imhof, A. van Blaaderen, Langmuir 2003, 19, 6693-6700.
14. a) D. Zins, V. Cabuil, R. Massart, J. Mol. Liq. 1999, 83, 217-232; b) M. H. Sousa, F. A. Tourinho, J. Depeyrot, G. J. da Silva, M. C. F. L. Lara, J. Phys. Chem. B 2001, 105, 1168-1175.
15. a) N. A. M. Verhaegh, A. van Blaaderen, Langmuir 1994, 10, 1427-1438; b) A. van Blaaderen, V. Vrij, Langmuir 1992, 8, 2921-2931.
16. a) S. Santra, P. Zhang, K. Wang, R. Tapec, W. Tan, Anal. Chem. 2001, 73, 4988-4993; b) K. P. Mcnamara, Z. Rosenzweig, Anal. Chem. 1998, 73, 4853-4859.

### Part Two

### Detailed Description

Part Two relates to Part One but further describes, for example, a surface charge effect of particles on cell-incorporation and also, for example, a gene delivery application. In particular, part two further describes the preparation of novel multifunctional nanomaterials of silica-coated magnetic core-shell nanoparticles, MNP@SiO₂, and modification the surface of nanoparticles with various charged chemicals. These modified nanoparticles showed a surface charge effect on the efficiency of incorporation and the localization into cells, which could be verified with a conventional confocal laser scanning microscope (CLSM) through detecting the fluorescent intensity from, for example, rhodamine β isothiocyanate (RITC) dye, chemically imbedded in the shell which can be, for example, a silica shell. The positively charged magnetic nanoparticle could be hybridized with plasmid DNA and this hybrid complex showed very high transfection efficiency (about 95%), rendering it a good candidate as an effective and convenient gene delivery vesicle. For the ultimate goal of nanomaterials in many interesting biological applications with safety and reliability, a useful surface modification method is described to introduce silica shells on magnetic nanoparticles with various organosilicon compounds (RSi(OR′)₃) and the valuable uses of surface-modified MNP@SiO₂(RITC)s such as specific bio-imaging and gene delivery applications.

In general, it is desired to coat or derivatize the surface with a variety of biomolecules or biologically active agents. DNA, genes, oligonucleotides, RNA, peptides, proteins, antibodies, drugs, and carbohydrates are leading examples. In many cases, specific recognition is desired. Generally, a negative surface charge can be used to attract a positively charged moiety, and a positive surface charge can be used to attract a negatively charged moiety. The surface charge density can be well-controlled to achieve the desired derivatization and release properties.

### Part Two

### Working Examples

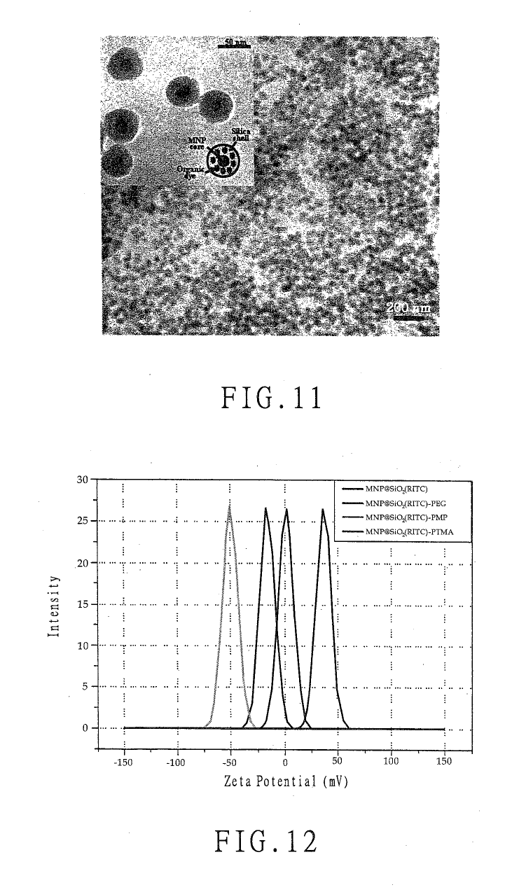

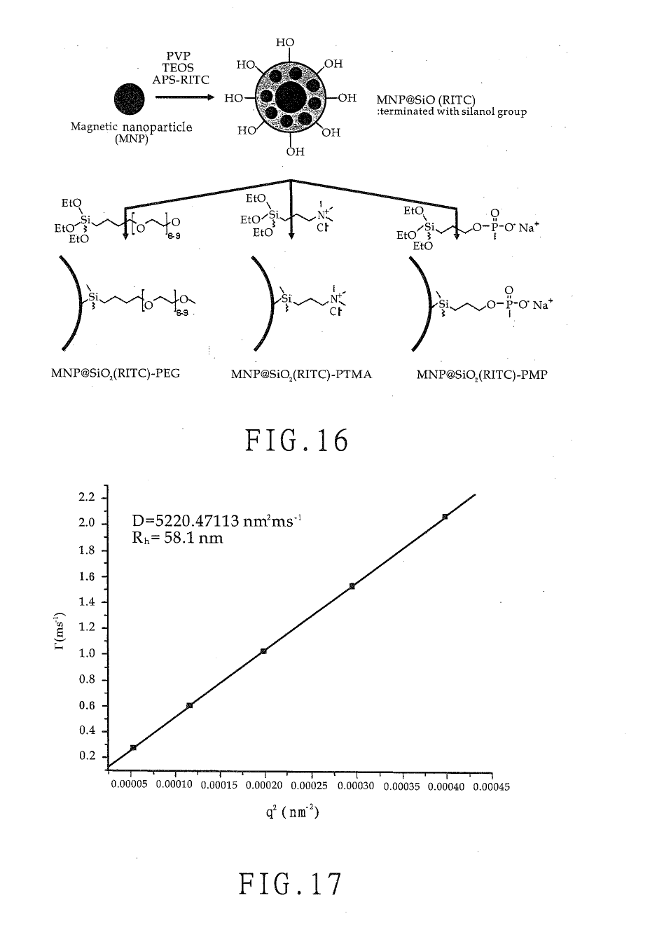

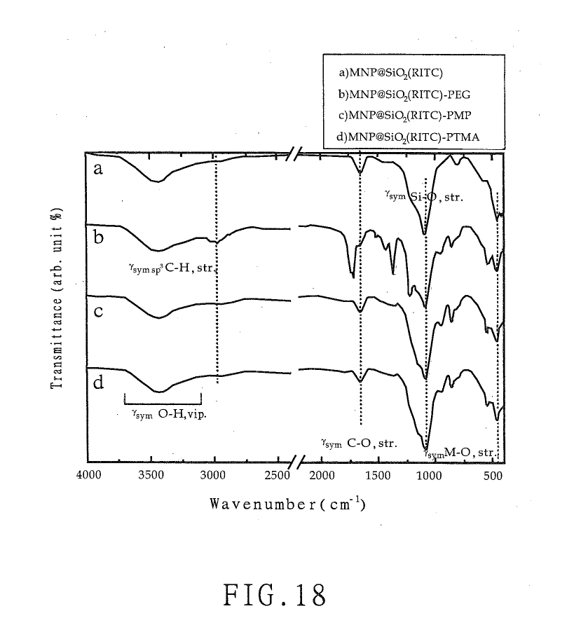

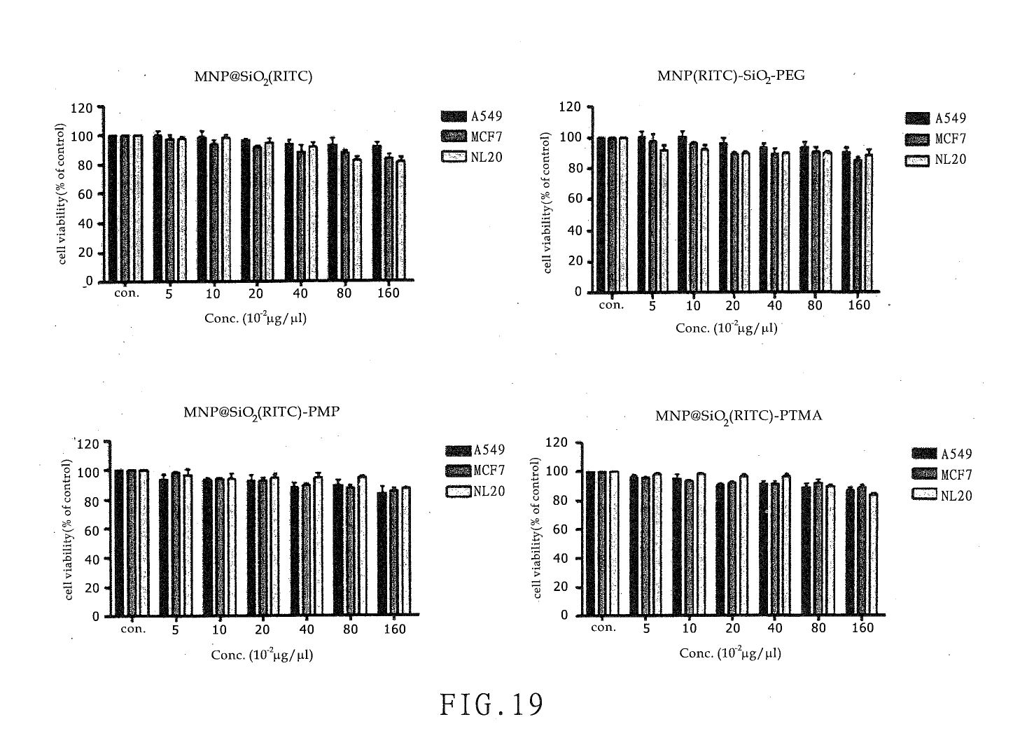

MNP@SiO₂(RITC)s (orange emission color, λ_{max(em.)}=555 nm) with an average size of 58 nm, confirmed by TEM and dynamic light scattering (DLS) experiments (FIGS. 1 and 18), were prepared as reported and used for the surface modification studies. Various trialkoxysilane derivatives were introduced on the silica shell surface by base-catalyzed condensation reaction with surface Si—OH functional groups as depicted in FIG. 16. Although there are no general limitations in the choosing of trialkoxysilane derivatives, neutral 2-[methoxy(polyethyleneoxy)propyl]-trimethoxysilane, (MeO)₃Si-PEG, anionic [3-(trihydroxysilyl)propyl]methylphosphonate sodium salt, (MeO)₃Si-PMP, and cationic N-trimethoxysilylpropyl-N,N,N-trimethylammonium chloride, (MeO)₃Si-PTMA, were employed to prepare surface-modified nanoparticles for the water-based biological applications. Attachment of Si-moieties on the surface did not significantly change the size of nanoparticles as observed in TEM measurements. Surface modifications were qualitatively confirmed by monitoring the changes of specific IR peaks from the ions and molecules on the surface (See FIG. 19) as well as measuring surface zeta potential values of the modified MNP@SiO₂(RITC)s (FIG. 12). As expected, MNP@SiO₂(RITC)-PMP has the highest negative surface charges and MNP@SiO₂(RITC)-PTMA has the highest positive charges, while unmodified MNP@SiO₂(RITC) and MNP@SiO₂(RITC)-PEG exhibit slightly negative and almost zero charges, respectively.

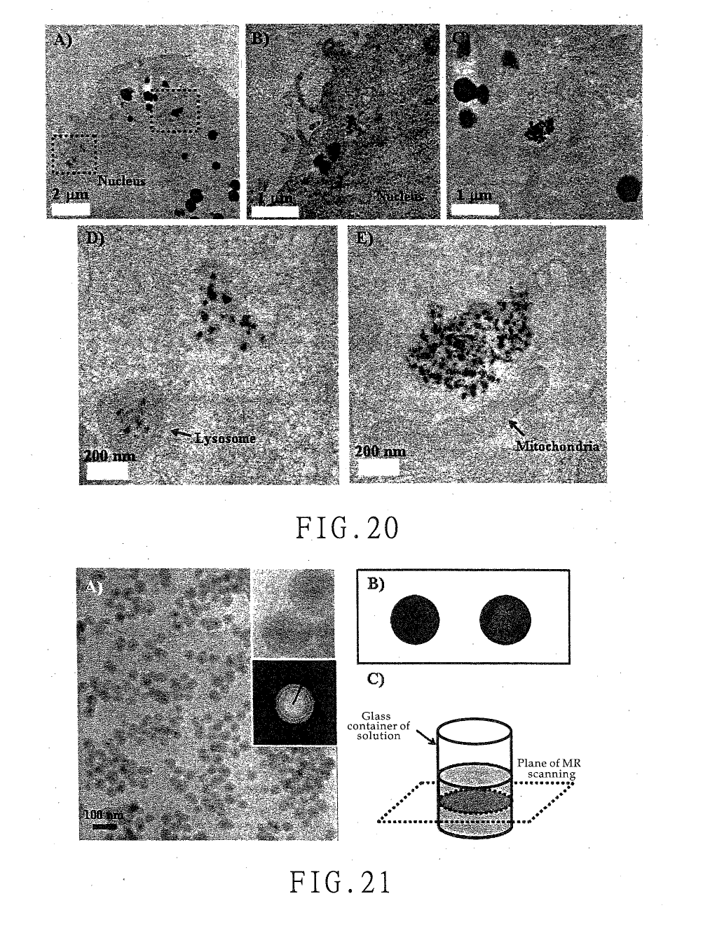

In order to use these modified nanoparticles for biological applications, cytotoxicity of various MNP@SiO₂(RITC) derivatives was evaluated on mammalian cells including breast cancer cell (MCF-7), lung normal cell (NL20), and lung adenocarcinoma cell (A549) with a 3-(4,5-dimethylthiazol-2-yl)-2,5-diphenyltetrazolium bromide (MTT) assay (See FIG. 20). In these tests, the cell viability was maintained in the range of greater than 85% in all groups indicating that the surface modified MNP@SiO₂(RITC)s do not have obvious cytotoxicity towards various cells at the level of a few tens of micrograms (80 μg mL⁻¹) within 48 h.

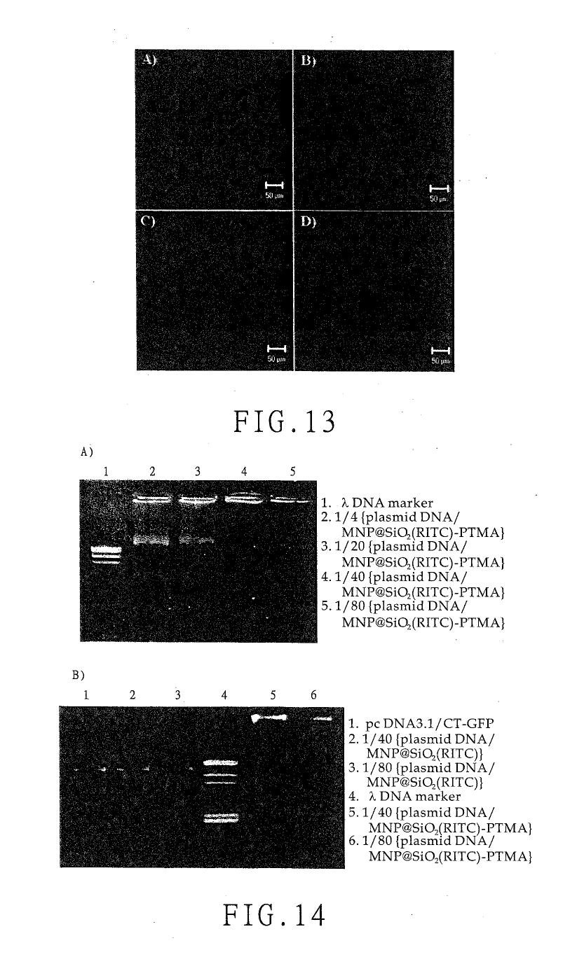

The incorporation efficiencies of various charged nanoparticles by the A549 cell were investigated under the same culturing conditions (FIG. 13). Based on the fluorescence intensities from incorporated MNP@SiO₂ derivatives in the cells measured by confocal laser scanning microscope (CLSM), MNP@SiO₂(RITC)-PEG exhibited the highest incorporation efficiency presumably due to the biocompatibility of PEG units as reported (reference 25). The relative intensities of fluorescence from the cells incorporating MNP@SiO₂ derivatives were observed in the order of MNP@SiO₂(RITC)-PEG >>MNP@SiO₂(RITC)-PTMA≈MNP@SiO₂(RITC)>MNP@SiO₂(RITC)-PMP. Although the reason for this trend is not clear at this stage, and the present embodiments are not limited by scientific theory, electrostatic interactions between surface charges of MNP@SiO₂ derivatives and mainly negative lipid double layer structure of the outer cell membrane seem to play a major role.

Once the stability of surface modified nanoparticles was confirmed and their cytotoxicities and preliminary incorporation effects on various cells were studied, their application as a gene delivery carrier was investigated (reference 26). Although many reported bio-carriers such as liposome, micelle, or dendrimer have their own advantages on drug or gene delivery, several significant limitations hamper their actual applications (reference 27). The liposome and micelle suffer from intrinsic chemical instability that limits the routes of administration and shelf life. In the blood, they are also known to be susceptible to disintegration through biochemical attack from high-density lipoproteins (reference 28). Dendrimer has low blood stability and is quickly eliminated through the kidney and liver. Furthermore, due to its extremely small size (typically <10 nm), it can pass through small intercellular openings and thus distribute non-specifically in healthy tissue (reference 29). Therefore, more biocompatible silica-coated multifunctional core-shell nanoparticles have recently been intensively studied.

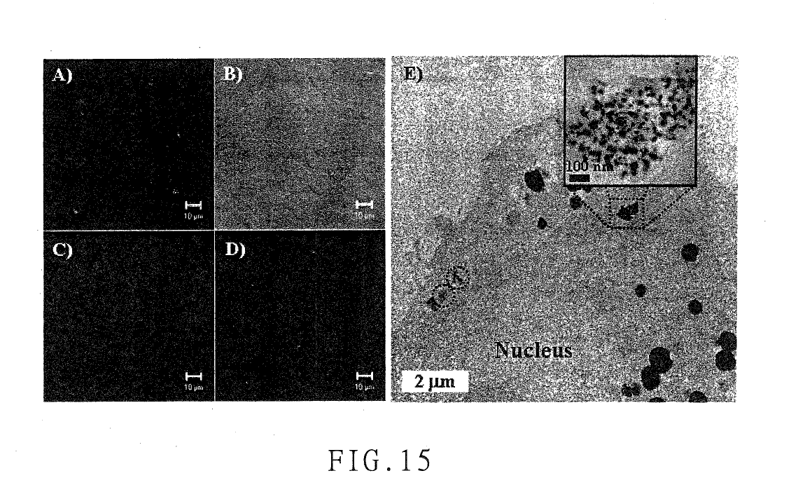

Positively charged MNP@SiO₂(RITC)-PTMA was hybridized with negatively charged plasmid DNA which could not be transfected into a cell by itself due to the charge repulsion by the negative character of the cell membrane (reference 32). To find the most suitable composition ratio making neutral or positive surface charges of hybridized complexes, various amount of plasmid DNA (pcDNA3.1/CT-GFP) were mixed with certain amount of MNP@SiO₂(RITC)-PTMA at pH ˜7.4, and agarose gel electrophoresis experiments (1.0%, 45 mM TBE buffer) were performed for 1 h at 100 V. The mixed samples of plasmid DNA (pcDNA3.1/CT-GFP) and MNP@SiO₂(RITC)-PTMA with various weight ratio from 1/4 to 1/80 produced typical electrophoretic gel shift bands as shown in FIG. 14A. When a relatively large amount of plasmid DNA was mixed, free plasmid DNAs not bound on the surface of MNP@SiO₂(RITC)-PTMA showed gel shift bands (lanes 2 and 3 in FIG. 14A). However, no gel shift bands from free plasmid DNA were observed when the amount of plasmid DNA was small enough to completely form hybrid complexes (lanes 4 and 5 in FIG. 14A). The surface charge effect was confirmed by employing neutral MNP@SiO₂(RITC) instead of positively charged MNP@SiO₂(RITC)-PTMA with a small ratio of plasmid DNA to be completely hybridized; the weight ratio of plasmid DNA/magnetic nanoparticle was 1/40 or 1/80. As clearly seen in lanes 2 and 3 in FIG. 14B, gel shift bands from free plasmid DNA were developed, confirming that the hybridization of negatively charged plasmid DNA with neutral MNP@SiO₂(RITC) did not occur. In the same electrophoresis experiment conditions, as expected, gel shift bands from free plasmid DNA were not observed in the samples containing positively charged MNP@SiO₂(RITC)-PTMA (lanes 5 and 6 in FIG. 14B). These results demonstrated that all the plasmid DNA could bind on the surface of MNP@SiO₂(RITC)-PTMA to form stable hybrid complex when the ratio of (plasmid DNA)/{MNP@SiO₂(RITC)-PTMA} was smaller than 1/40 (w/w).

To investigate the transfection efficiency of the (plasmid DNA)/{MNP@SiO₂(RITC)-PTMA} hybrid system, the solution of 1 μg DNA (in 1 μL H₂O) and 40 μg MNP@SiO₂(RITC)-PTMA (in 10 μL H₂O) was added to the A549 cells on 6-well plates {5×10⁴ cells per well in 2.0 mL Roswell Park Memorial Institute (RPMI 1640) medium without fetal bovine serum (FBS)} and cells were incubated for 4 h at 37° C. The cells were then washed with PBS (pH 7.2 phosphate buffered saline) and cultured in 2 mL of RPMI with 10% FBS and antibiotics for 2 days. As known for the general mechanism of DNA transfection (references 30, 31), the plasmid DNA could be released from nanoparticle surface due to the pH change in the cytoplasm, which induced the protonation of many negative sites in DNA to weaken the interaction with the cationic nanoparticle surface. The released plasmid DNA in our system was pcDNA3.1/CT-GFP which could develop green fluorescence when it was successfully delivered, while the delivery carrier MNP@SiO₂(RITC)-PTMA residing in the cytoplasm generated its own orange fluorescence (FIG. 15). All the cells showed a green emission in the whole area of cytoplasm, as expected, and a localized orange emission from a relatively small amount of MNP@SiO₂(RITC)-PTMA, which was clearly shown in the merged image of FIG. 15d and cross-sectioned TEM image of FIG. 15e. A most promising point from this preliminary gene delivery experiment was the high yield of transfection (almost 95%, flow cytometer, Becton Dickinson, Calif., USA), and the localized distribution of nanoparticles in the nearby subcelluar organelles such as lysosome and mitochondria (FIG. 21). This observation also confirms that the MNP@SiO₂(RITC)s do not have acute cytotoxicity because these organelles were known to disappear rapidly upon cell death.

In summary, it has been demonstrated that the surface of magnetic core-shell nanoparticles, MNP@SiO₂, were modified with various charged chemicals and the modified nanoparticles showed surface charge effect on the efficiency and the localization of incorporation into cells, which could be identified with conventional confocal laser scanning microscope (CLSM) through the detection of fluorescent intensity from RITC dye imbedded in the silica shell. These charge-dependent and functionality-dependent phenomena for the incorporation of nanoparticle are expected to give critical clues for controlling the site-specific targeting and staining applications. Furthermore, the positively charged magnetic nanoparticle, MNP@SiO₂(RITC)-PTMA, could be hybridized with plasmid DNA for the delivery into cells and a very high transfection efficiency was observed. It is hoped that the hybrid complex of plasmid DNA/MNP@SiO₂(RITC)-PTMA prepared from this convenient procedure could find many biological gene delivery applications, where efficient and reproducible transfection with an easy monitoring tool such as fluorescence detection is needed.

### Experimental Section

RITC was purchased from Fluka (Switzerland). Silicon compounds such as APS, TEOS, (MeO)₃Si-PEG, (MeO)₃Si-PMP, and (MeO)₃Si-PTMA were from Gelest (Morrisville, Pa., USA). These chemicals were used without further purification. MCF-7, NL20, and A549 were ordered from American Type Culture Collection (ATCC, Manassas, Va.).

Preparation of MNP@SiO₂(RITC): Cobalt ferrite solution (34.7 mL; 20 mg MNP mL⁻¹ solution in water) was added to polyvinylpyrrolidone solution (PVP; 0.65 mL; Mᵣ 55,000 Da; 25.6 g L⁻¹ in H₂O), and the mixture was stirred for 1 day at room temperature. The PVP-stabilized cobalt ferrite nanoparticles were separated by addition of aqueous acetone (H₂O/acetone=1/10, v/v) and centrifugation at 4000 rpm for 10 min. The supernatant was removed and the precipitated particles were redispersed in ethanol (10 mL). Multigram-scale preparation of PVP-stabilized cobalt ferrite nanoparticles was reproduced in this modified synthetic method. Triethoxysilane modified by Rhodamine β isothiocyante (RITC) was prepared from 3-aminopropyltriethoxysilane (APS) and rhodamine β isothiocyante under nitrogen using a standard Schlenk line technique (reference 32). A mixed solution of TEOS and RITC-modified triethoxysilane (TEOS/RITC-silane=0.3/0.04, molar ratio) was injected into the ethanol solution of PVP-stabilized cobalt ferrite. Polymerization initiated by adding ammonia solution (0.86 mL of 30 wt % NH₄OH) as a catalyst produced cobalt ferrite-silica core-shell nanoparticles containing organic-dye. These nanoparticles were dispersed in ethanol and precipitated by ultra-centrifugation (18,000 rpm, 30 min).

Modification of MNP@SiO₂(RITC) with various Si-compounds: The purified core-shell nanoparticles (45 mg) were redispersed in absolute ethanol (10 mL) and then 0.5 mmol of Si-compounds ((MeO)₃Si-PEG, 275 mg; (MeO)₃Si-PMP, 119 mg; (MeO)₃Si-PTMA, 128 mg, respectively) was added and stirred for 2 h at pH about 12 (adjusted with NH₄OH), heated to ˜60° C. for less than 5 min, and cooled to ˜30° C. The remaining silanol groups were quenched with a mixture of methanol (20 mL) and trimethylchlorosilane (11 mg, 0.1 mmol) basified with solid TMAH pentahydrate (0.5 g), and then stirred again for 2 h. The solution was heated to ˜60° C. for 30 min, and then left at room temperature for 2 h while stirring in a N₂ atmosphere. The resulting mixture was precipitated by ultra-centrifugation and purified nanoparticles were redispersed in PBS buffer solution for the applications.

Cell culture: MCF-7 cells were grown in Dulbecco's Modified Eagle Medium (DMEM, 2 mL, culture media; Cambrex Bio Science, Walkersville, Md.) containing with 10% FBS (v/v), 0.5% gentamicin (v/v), and nanoparticle solution (2 mg mL⁻¹; 40 μl). NL-20 and A-549 were grown in RPMI (culture media; Cambrex Bio Science) under the same conditions. All cells were cultured in Lab-Tek glass chamber slide (Nalge Nunc International, Naperville, Ind.) in order to observe fluorescence emission by confocal laser scanning microscopy (CLSM).

Agarose gel electrophoresis: Nanoparticles and plasmid DNA (pcDNA3.1/CT-GFP; Invitrogen, USA) were hybridized with various DNA/nanoparticles ratios in 10 mM HEPES buffer (pH 7.4) and 100 mM CaCl₂ (aqueous). After incubation at 4° C. for 4 h, hybridized nanoparticles with plasmid DNA were analyzed on 1.0% agarose gels, stained with ethidium bromide in TBE buffer (44.5 mM Tris base, 4.5 mM boric acid, 0.5 M EDTA, pH 7.8) and photographed under UV light (Japan, FUJI Film, LAS3000).

Protocol of Transmission Electron Microscope: To study the metabolism of nanoparticles, treatment cells were fixed with 1% glutaaldehyde and 1.5% paraformaldehyde in 0.1 M phosphate buffer, pH 7.2 at 4° C. The samples were then washed in PBS followed by washing in 0.1 M cacodylate buffer, pH 7.2 and post-fixed in 1% osmium tetraoxide in 0.1 M cacodylate buffer for 1.5 hour at room temperature. The samples were then washed briefly in dH₂O and dehydrated through a graded ethanol series and infiltrated by using of propylene oxide and EPON epoxy resin (EmBed 812, Electron Microscopy Sciences), and finally embedded with only epoxy resin. The epoxy resin mixed samples were loaded into capsules and polymerized at 60° C. for 24 hours. Thin sections were made using a RMC MT-X ultramicrotome and collected copper grids and not stained any reagent for detecting of nanoparticles into the cells. Images were collected using a JEOL (JEM-1011) transmission electron microscope at 80 kV with a GATAN digital camera.

CLSM: Fluorescence image of organic dye-labeled nanoparticles were obtained with a Zeiss LSM 510 CLSM equipped with a computer-controlled scan stage. An argon laser for RITC excitation at 532 nm was used for imaging.

FIG. 18 and DLS Data: Dynamic light scattering measurements were performed using a UNIPHASE He—Ne laser operating at 632.8 nm. The maximum operating power of the laser was 30 mW. The detector optics employed optical fibers coupled to an ALV/SO-SIPD/DUAL detection unit, which employed an EMI PM-28B power supply and ALV/PM-PD preamplifier/discriminator. The signal analyzer was an ALV-5000/E/WIN multiple tau digital correlator with 288 exponentially spaced channels. Its minimum real sampling time is 10⁻⁶ s and a maximum of about 100 s. A lens with a focal length of 200 mm narrowed the incident beam to reduce the thermal lensing effect and to increase the coherence area. The scattered beam passed through two pin holes before reaching the PMT. A scattering cell (10 mm diameter cylindrical) was placed in a temperature controlled bath of index matching liquid, decaline. All of the experiments were performed at 25±0.1° C.

FIG. 20, MTT Assay Data: The cells were incubated in a 96-well plate. At the end of the incubation period, 3(4,5-dimethylthiazol-2-yl) 2,5-diphenyltetrazolium bromide (MTT; 50 μL, Sigma-Aldrich) in PBS (0.2 mg mL⁻¹) was added to each well (final concentration of 0.4 mg mL⁻¹) and cultures were incubated in 5% CO₂ for 4 h at 37° C. Then the culture medium was carefully removed by pipetting and formazan crystals generated by dehydronase activity in mitochondria, which only occurs in living cells, were dissolved in DMSO for the analysis. After 10 min agitation on a shaker, absorbance was measured at 490 nm and 620 nm for test and reference solutions, respectively.

### PART II

### References

17. Dubertret, B.; Skourides, P.; Norris, D. J.; Noireaux, V.; Brivanlou, A. H.; Libchaber, A. Science 2002, 298, 1759; Thomas, M.; Klibanov, A. M. Proc. Natl. Acad. Sci. USA 2003, 100, 9138; Katz, E.; Willner, I. Angew. Chem. Int. Ed. 2004, 43, 6042.
18. Derfus, A. M.; Chan, W. C. W.; Bhatia, S, N. Nano Lett. 2004, 4, 11; Sayes, C. M.; Fortner, J. D.; Guo, W.; Lyon, D.; Boyd, A. M.; Ausman, K. D.; Tao, Y. J.; Sitharaman, B.; Wilson, L. J.; Hughes, J. B.; West, J. L.; Colvin, V. L. Nano Lett. 2004, 4, 1881.
19. Kondoh, M.; Araragi, S.; Sato, K.; Higashimoto, M.; Takiguchi, M.; Sato, M. Toxicol. 2002, 170, 111; Kirchner, C.; Liedl, T.; Kudera, S.; Pellegrino, T.; Javier, A. M.; Gaub, H. E.; Stölzle, S.; Fertig, N.; Parak, W. J. Nano Lett. 2005, 5, 331; Limaye, D. A.; Shaikh, Z. A. Toxicol. Appl. Pharmacol. 1999, 154, 59; Goyer, R. A. Am. J. Clin. Nutr. 1995, 61, 646s.
20. Correa-Duarte, M. A.; Giersig, M.; Kotov, N. A.; Liz-Marán, L. M. Langmuir 1998, 14, 6430; Shchipunov, Y. J. Colloid Interf. Sci. 2003, 268, 68.
21. Barbé, C.; Bartlett, J.; Kong, L.; Finnie, K.; Lin, H. Q.; Larkin, M.; Calleja, S.; Bush, A.; Calleja, G. Adv. Mater. 2004, 16, 1959; Nann, T.; Mulvaney, P. Angew. Chem. Int. Ed. 2004, 43, 5393.
22. Lu, Y.; Yin, Y.; Mayers, B. T.; Xia, Y. Nano Lett. 2002, 2, 183.
23. Ow, H.; Larson, D. R.; Srivastava, M.; Baird, B. A.; Webb, W. W.; Wiesner, U. Nano Lett. 2005, 5, 113; Graf, C.; Schärtl, W.; Fischer, K.; Hugenberg, N.; Schmidt, M. Langmuir 1999, 15, 6170.
24. Yoon, T. J.; Kim, J. S.; Kim, B. G.; Yu, K. N.; Cho, M. H.; Lee, J. K. Angew. Chem. Int. Ed. 2005, 44, 1068; Gu, H.; Ho, P.; Tsang, K. W. T.; Wang, L.; Xu, B. J. Am. Chem. Soc. 2003, 125, 15702; Wang, D.; He, J.; Rosenzweig, N.; Rosenzweig, Z. Nano Lett. 2004, 4, 409.
25. Zhang, Y.; Kohler, N.; Zhang, M. Biomaterials 2002, 23, 1553; Yamazaki, M.; Ito, I. Biochemistry 1990, 29, 1309.
26. Radu, D. R.; Lai, C.; Jeftinija, K.; Rowe, E. W.; Jeftinija, S.; Lin, V. S. Y. J. Am. Chem. Soc. 2004, 126, 13216; Esfand, R.; Tomalia, D. A. Drug Discovery Today 2001, 6, 427; Luo, D.; Saltzman, W. M. Nat. Biotechnol. 2000, 18, 893.
27. Martini, A.; Ciocca, C. Expert Opin. Ther. Patents 2003, 13, 1801; Emerson, O. L. Pharm. Sci. Technol. Today 2000, 3, 205; Oussoren, C.; Storm, G. Adv. Drug Delivery Rev. 2001, 50, 143; Kataoka, K.; Harada, A.; Nagasaki, Y. Adv. Drug Delivery Rev. 2001, 47, 113.
28. Allen, C.; Dos Santos, N.; Gallagher, R.; Chiu, G. N. C.; Shu, Y.; Li, W. M.; Johnstone, S. A.; Janoff, A. S.; Mayer, L. D.; Webb, M. S.; Bally, M. B. Biosci. Rep. 2002, 22, 225.
29. Jevprasesphant, R.; Penny, J.; Attwood, D.; D'Emanuele, A. J. Controlled Release 2004, 97, 259; Esfand, R.; Tomalia, D. A. Drug Discov. Today 2001, 6, 427.
30. Lu, Q.; Moore, J. M.; Huang, G.; Mount, A. S.; Rao, A. M.; Larcom, L. L.; Ke, P. C. Nano Lett. 2004, 4, 2473.
31. Roy, I.; Ohulchanskyy, T. Y.; Bharali, D. J.; Pudavar, H. E.; Mistretta, R. A.; Kaur, N.; Prasad, P. N. Proc. Natl. Acad. Sci. USA 2005, 102, 279; Brouet, A.; Sonveaux, P.; Dessy, C.; Balligand, J.; Feron, O. J. Biol. Chem. 2001, 276, 32663; Xu, J.; Liao, K. J. Biol. Chem. 2004, 279, 35914.
32. Verhaegh, N. A. M.; Van Blaaderen, A. Langmuir 1994, 10, 1427; Van Blaaderen, A.; Vrij, V. Langmuir 1992, 8, 2921.

### Part Three

### Part Three

### Detailed Description

In addition, the surface can be adapted to introduce functional groups which allow for covalent surface modification and organic synthesis, going beyond electrostatic attraction and ionic bonding. For example, the surface can be derivatized with a nucleophilic group such as, for example, a Lewis base group such as an amine group. The reactions of nucleophiles with electrophiles are well-known.

### Part Three

### Working Examples

It has been demonstrated that the organic dye-incorporated smart silica core-shell magnetic nanoparticles (MNP@SiO₂), detectible by fluorescence and MRI imaging, were fabricated using silicon compounds having dual-functionality (-PEG/NH₂) and the amine moieties on the nanoparticle surface were modified with maleimide functionality for specific covalent immobilization of biopolymers and bioactive small molecules. The sequence-independent immobilization of antibodies (Ab_{CD-10} or Ab_{HER-2}) was highlighted in this report. The Ab_{CD-10} modified magnetic nanoparticles {MNP@SiO₂(FITC)-Ab_{CD-10}} exhibited the specific recognition of floating tumor cells (SP2/0) and the possible applicability of cell separation by the application of an external magnetic field. The MNP@SiO₂(FITC)-Ab_{HER-2} specifically targeted the membrane of the adherent breast cancer cell (MCF-7) and the co-treatment with differently modified MNP@SiO₂(RITC) and MNP@SiO₂(FITC) nanoparticles which results in cellular uptake by endocytosis and cellular targeting, can be used for bio-imaging.

In this study, the silica shell of MNP@SiO₂ incorporated with organic dyes (rhodamine β isothiocyanate, RITC, orange color, λ_{max(em.)}=555 nm, or fluorescein isothiocyanate, FITC, green color, λ_{max(em.)}=518 nm) were modified with various functional organosilicon compounds (Si-compounds) such as (MeO)₃Si-PEG, {CH₃O(CH₂CH₂O)_{6˜9}CH₂CH₂CH₂Si(OCH₃)₃, 2-[methoxy(polyethyleneoxy)propyl]trimethoxysilane} and APS, {(MeO)₃Si-(CH₂)₃NH₂, 3-aminopropyltriethoxysilane}. The surface of the dual-fabricated core-shell nanoparticles presents two key functional groups-the PEG moiety that enhances biocompatibility in vivo and in vitro (reference 45), and an amine moiety to which the desired molecules or biopolymers could be added, which in turn increase the applicability of MNP. The amine site is the key surface-bound moiety for the immobilization of biomolecules (or antibody). Based on the complete characterization of amine concentration, it was determined that the PEG/amine dual modified MNP@SiO₂ {MNP@SiO₂(OD)-PEG/NH₂} has approximately 6.5±0.9 mmol·g⁻¹ amine and 3.9×10⁴ amine moieties per core-shell nanoparticle by using the standard Fmoc quantification protocol. In order to create the modular system for the specific immobilization of various biomolecules or biopolymers, a maleimide moiety was introduced through covalent linkage with the amine moiety on the surface of a smart core-shell nanoparticle (FIG. 27). A maleimide moiety can selectively immobilize thiol-containing biomolecules and/or biopolymers. The freely accessible maleimide moieties on the MNP@SiO₂(OD)-PEG/NH₂ surface were quantified by the treatment with Fmoc-protected aminoethanethiol in accordance with the Fmoc quantification protocol. Using this method, it was determined that 55 mol % of maleimide (3.6±0.6 mmol·g⁻¹) was available on the surface for selective immobilization of thiol-containing biomolecules. Therefore, it was concluded that it was important to cap the unreacted amine moieties on the surface with acetyl groups by incubation with acetic anhydride. The complete control and characterization of surface-bound PEG/amine moieties on our dual-fabricated nanoparticles will allow the development of reproducible protocols with quality control, which is critical for its biomedical application.

The transmission electron microscope (TEM) images of synthetic smart MNP@SiO₂(FITC) revealed spherical structures having a total size of 60 nm. Further, as confirmed by electron beam diffraction pattern, the crystallinity of the MNP core was not altered during the preparation of silica shell (FIG. 21A). The prepared core-shell nanoparticles were homogeneously dispersed in an aqueous solution. The stability and homogeneity were confirmed by dynamic light scattering (DLS) experiment, and the magnetic property was characterized by a vibrato sample magnetometer (VSM). FIG. 21B shows T₂ weighted spin-echo MR images of MNP@SiO₂(FITC) nanomaterial in aqueous solution at 4.7 Tesla and the contrast difference corresponded to the concentration of the magnetic nanoparticles. Typically, paramagnetic iron oxide is shown negative contrast sign in T₂ relaxation time (reference 37, 46).

Following the confirmation of homogeneous dispersion in an aqueous system as well as the stability and non-toxic nature of our smart magnetic core-silica shells, it was further investigated the possibility of the application of nanoparticles in the biomedical field, particularly for specific targeting, cell sorting, and bio-imaging. The model system chosen was the floating tumor cell (SP2/0, leukemia) having CD-10 receptors on its outer cell membrane, and CD-10 antibody (Ab_{CD-10}) was immobilized on smart organic dye incorporated magnetic core-silica shell nanomaterials. Briefly, MNP@SiO₂(FITC)-Ab_{CD-10} was prepared for the experiment. The reported immobilization method, which is a random crosslinking of Ab_{CD-10} on the surface amine moieties using glutaraldehyde (reference 47), resulted in immobilization of the antibody onto the nanoparitcle; however the Ab_{CD-10} immobilized nanoparticle had several limitations such as high aggregation and low reproducibility. In comparison to the random immobilization of antibodies, our smart magnetic core-silica shell nanoparticles are capable of qualitative and specific immobilization through maleimide moieties on the silica shell. To establish a general protocol for the immobilization of any type of antibody in the absence of sequence information, sulfhydryl residues were introduced into the antibody molecules for conjugation with maleimide-modified MNP@SiO₂(FITC)-PEG/NH₂ by reducing indigenous disulfide linkages in the antibody hinge region. Reduction with 2-mercaptoethylamine (MEA) leads to cleavage of the disulfide bonds that hold the heavy chains together; however, the disulfide bonds between the heavy and light chains remains unaffected. The sulfhydryl groups produced by this reduction can couple with our smart magnetic core-silica shell nanoparticles without blocking the antigen binding site. (See experimental section for detailed procedure).

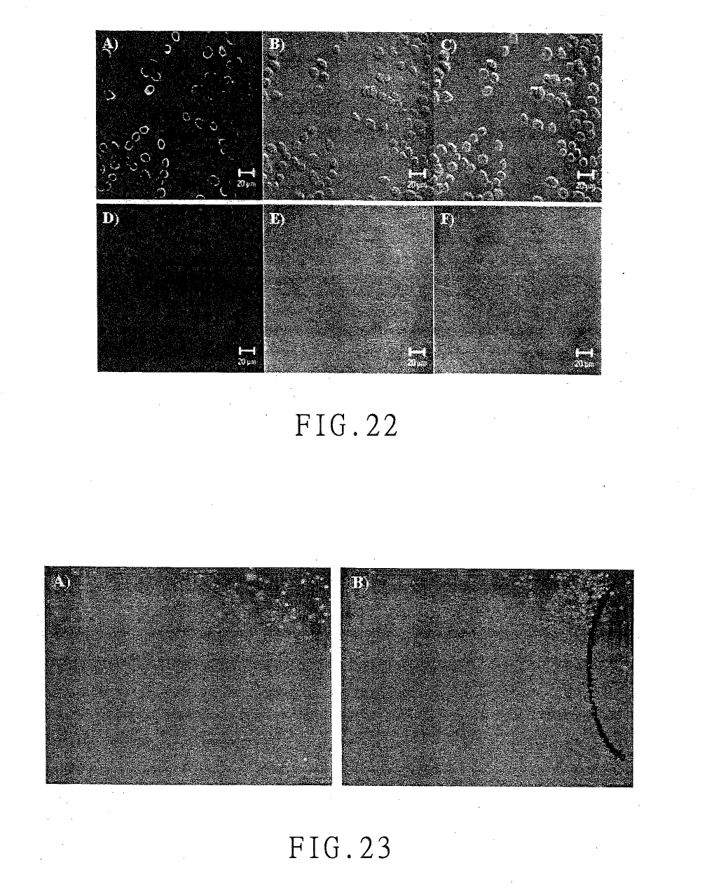

Specific targeting was examined by the incubation of floating tumor SP2/0 cell and a lung cancer cell (A549) with MNP@SiO₂(FITC)-Ab_{CD-10} (reference 48). As shown in FIG. 22, CLSM images confirmed the specific targeting of SP2/0 leukemia cells. On the other hand, the lung cancer cell (A549) lacking CD-10 receptors that were used as negative control was not targeted indicating the absence of nonspecific targeting. Based on the specific targeting ability and sequence-independent antibody immobilization protocol, it was confirmed that our smart magnetic core-silica shell nanoparticle can be applied to any antigen-antibody specific targeting in biomedical systems. Specific targeting ability of the SP2/0 cell allowed the examination of the next stage of biomedical application, cell sorting. Optical microscope images in FIG. 23 were taken from the moving picture focused at the bottom of the container flask before and after an external magnetic field was applied using a commercial Nd—Fe—B magnet (˜0.3 Tesla) on the inside of the petri-dish (red-dot circle position in FIG. 23B), containing SP2/0 cells specific targeted with MNP@SiO₂(FITC)-Ab_{CD-10}. The local accumulation of the floating cells was possible only if the cells were targeted by our smart nanomaterials and they in turn responded to the externally applied magnetic field. Changing the location of the external magnet resulted in a change in the direction of cell movement. Dramatic images of the cell movement during the cell sorting experiment can be observed. One can clearly observe cell sorting by the application of an external magnetic field via antibody-specific targeting using biocompatible magnetic core-silica shell nanomaterials.

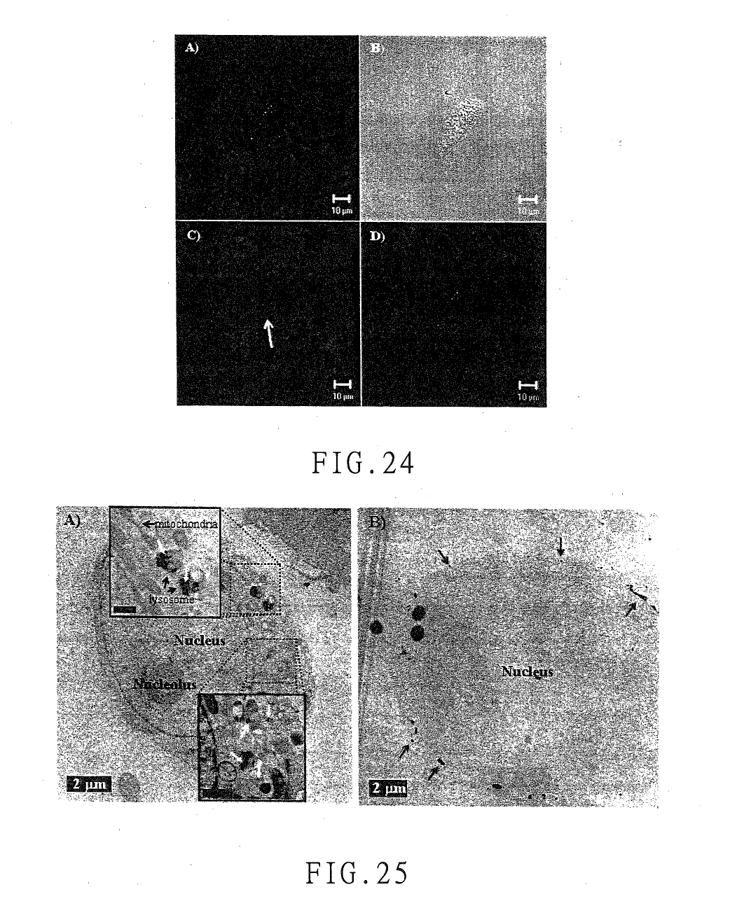

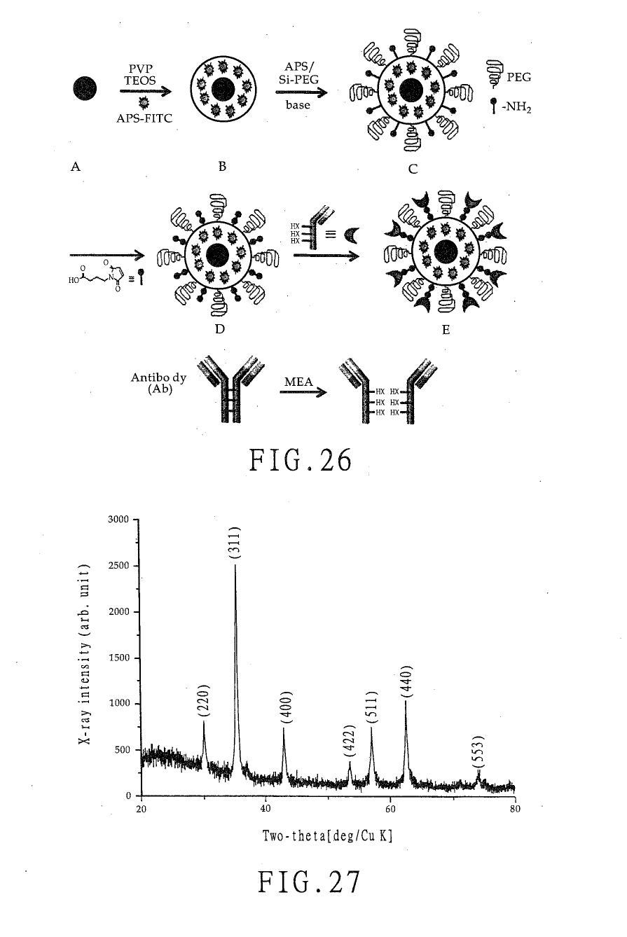

Furthermore, the smart magnetic core-silica shell nanoparticle was applied to the field of bio-imaging. The test system of choice was HER-2 antibody (Ab_{HER-2}) which is generally used for specific targeting to a receptor on breast cancer cell (MCF-7) membrane surface (reference 49), and MNP@SiO₂(FITC)-Ab_{HER-2} was successfully prepared using the same protocols as those used for the preparation of Ab_{CD-10}. This validated the generality of the system for immobilization of any type of antibody. It is reported that the unmodified MNP@SiO₂ nanomaterials were internalized into the cells by endocytosis (reference 50). The green color MNP@SiO₂(FITC)-Ab_{HER-2} and unmodified red nanoparticles {MNP@SiO₂(RITC)} could be specifically located on the membrane surface and in the cytoplasm of MCF-7 cells, respectively (FIG. 24). The MNP@SiO₂(FITC)-Ab_{HER-2}s were specifically localized on the cell membrane (FIG. 24A) and unmodified MNP@SiO₂(RITC) nanoparticles at the cytoplasm of cell (FIG. 24C). The internalized and membrane targeted core-shell nanoparticles of the MCF-7 cell were confirmed by TEM. Electron micrographs of the cells provided direct evidence that a large number of MNP@SiO₂(RITC) were endocytosed by MCF-7 cell (FIG. 25). It is noteworthy that the core-shell nanoparticles were entered into the lysosome and loaded at the cytoplasm (white arrows in inset images of FIG. 25A, See also FIG. 31 for enlarged micrographs). Furthermore, MNP@SiO₂(FITC)-Ab_{HER-2}s specifically targeted on the MCF-7 cells was revealed as black dots (red arrows in FIG. 25B) onto the membrane. Given the fact that these organelles disappear rapidly upon cell death, these results strongly suggested that the MNP@SiO₂(RITC)s and MNP@SiO₂(FITC)-Ab_{HER-2}s were not cytotoxic in vitro. Based on this result, our system can be applied to not only specific targeting and cell sorting but also intracellular compartment localization for bio-imaging.

In summary, the MNP@SiO₂(OD), which can be detected by fluorescence and MRI imaging, can be fabricated using silicon compounds having dual functionality (-PEG/NH₂). The amine moieties on the nanoparticle surface were coated with maleimide moieties for specific covalent immobilization of biopolymers as well as bioactive small molecules. In this report, the systematic antibody immobilization (Ab_{CD-10} or Ab_{HER-2}) was focused upon by a sequence-independent protocol, and the Ab_{CD-10} immobilized nanoparticles, MNP@SiO₂(FITC)-Ab_{CD-10}, exhibited the specific recognition of floating tumor cells and the possibility of cell separation by the application of an external magnetic field. The MNP@SiO₂(FITC)-Ab_{HER-2}s also specifically targeted the breast cancer adherent cells, and the co-treatment of MNP@SiO₂(RITC) nanoparticles, which results in their cellular uptake by endocytosis, can be used for biomedical imaging, and their specific location was confirmed by TEM measurement. Smart nanosystem materials detectable by fluorescence (tissue determination) and magnetic properties (nondestructive inspection) are currently under investigation of intelligent drug delivery system for real targeting and as a therapy for breast cancer cells in in vivo mouse models. These nanomaterials can be used in a number of biomedical applications in nano-biotechnology such as targeting, bio-imaging, cell sorting, drug delivery, and therapy systems.

### Experimental Section

FeCl₃.6H₂O, CoCl₂.6H₂O, Fe(NO₃)₃.9H₂O, anhydrous DMF, piperidine, 2-mercaptoethylamine (MEA), EDTA, and diisopropylethylamine were purchased from Sigma-Aldrich (St. Louis, Mo.). RITC, FITC, and maleimidobutric acid were from Fluka (Switzerland). Tri(2-carboxyethyl)phosphine hydrochloride (TCEP) and Sephadex G-25 were from Molecular Probes (Eugene, Oreg.). FmocCl, N-hydroxybenzotriazole (HOBt), and PyBOP were purchased from Novabiochem (Switerland). Silicon compounds such as APS, tetraethylorthosilicate (TEOS), and (MeO)₃Si-PEG were from Gelest (Morrisville, Pa., USA). All antibodies were purchased from Santa Cruz Biotechnology, Inc (Santa Cruz, Calif.). These chemicals were used without further purification.

Preparation of MNP@SiO₂(FITC)-PEG/NH₂: Cobalt ferrite solution (34.7 mL; 20 mg·mL⁻¹ solution in water) was added to polyvinylpyrrolidone solution (PVP; 0.65 mL; Mᵣ 55,000 Da; 25.6 g·L⁻¹ in H₂O), and the mixture was stirred for 1 day at room temperature. The PVP-stabilized cobalt ferrite nanoparticles were separated by addition of aqueous acetone (H₂O/acetone=1/10, v/v) and centrifugation at 4000 rpm for 10 min. The supernatant was removed and the precipitated particles were redispersed in 10 mL ethanol. Multigram-scale preparation of PVP-stabilized cobalt ferrite nanoparticles was reproduced in this modified synthetic method. FITC-modified triethoxysilane was prepared from APS and FITC under nitrogen using a standard Schlenk line technique (reference 42, 51). A mixed solution of TEOS and FITC-modified triethoxysilane (TEOS/FITC-silane molar ratio=0.3/0.04) was injected into the ethanol solution of PVP-stabilized cobalt ferrite. Polymerization initiated by adding ammonia solution (0.86 mL; 30 wt % by NH₃) as a catalyst produced cobalt ferrite-silica core-shell nanoparticles containing organic-dye. The harvested core-shell nanoparticles (370 mg) by centrifugation (18,000 rpm, 30 min) were redispersed in basic (pH ˜12, adjusted by NH₄OH) 50 mL ethanol, added with (MeO)₃Si-PEG (275 mg, 0.5 mmol) and APS (22 mg, 0.1 mmol), and stirred for 2 hr at room temp. in N₂ atmosphere. It was isolated from unreacted Si compounds by centrifugation and washed with EtOH for several times. (MNP was mixed with MeOH and separated by centrifugation at 13,000 rpm for 20 min. Supernatant was removed and MeOH was added again. Precipitated particles were redispersed by sonication for 20 min. Repeat this process several times.) Finally, it was redispersed in anhydrous 20 mL DMF solution.

Quantitative analysis of amine moieties on MNP@SiO₂(FITC)—PEG/NH₂: MNP@SiO₂(FITC)-PEG/NH₂ solution (3.2 mL; PEG/NH₂ molar ratio=5:1, 22.9 mg·mL⁻¹ in anhydrous DMF) was added to solution of FmocCl (0.15 g, 0.4 mmol) in anhydrous DMF (5 mL). The reaction mixture stirred overnight under nitrogen at room temperature. Reaction mixture was transferred to 6 pre-weighted Eppendorf tubes, followed by washing with MeOH for several times. After thorough washing, precipitated MNPs with complete Fmoc protection on amine moieties were dried under vacuum overnight. After weighing these MNPs, 0.8 mL of DMF was added and redispersed by sonication. 0.2 mL of piperidine was added and sonicated for 20 min. Amine quantification was performed using standard Fmoc quantification protocol by the detection with UV absorption of supernatant at λ=300 nm after centrifugation at 13,000 rpm for 20 min. The extinction coefficient at this wavelength is 7800 mol⁻¹·dm³·cm⁻¹.

Preparation and quantification of smart silica core-shell magnetic nanoparticles: MNP@SiO₂(FITC)-PEG/NH₂ solution (36.5 mL; Si-PEG/APS molar ratio=5:1, 22.9 mg·mL⁻¹ in anhydrous DMF, amine concentration on MNP=6.5 mmol·g⁻¹) was added to solution of maleimidobutric acid (0.96 g, 0.524 mmol, Fluka) and PyBOP (0.43 g, 0.826 mmol, Novabiochem) and HOBt (0.19 g, 1.4 mmol) in 5 mL of anhydrous DMF. Reaction mixture was added with freshly distilled diisopropylethylamine (0.2 mL) and stirred under nitrogen at room temperature overnight. Reaction mixture was transferred to a number of 40 Eppendorf tubes and each reaction mixture was washed by DMF for several times. After final washing, prepared MNP particle was redispersed in 0.8 mL of DMF through sonication for 20 min and can be stored in a desiccator at room temperature with light protection. To quantify the active maleimide moieties on MNPs, the following procedure was performed. 8 Eppendorf tubes charged with a 0.8 mL solution of MNP@SiO₂(FITC)-PEG/NH-maleimide in DMF was added with 0.2 mL of cocktail solution which contained Fmoc-protected 2-aminoethanethiol (50 mg, 0.167 mmol), sodium methanolate (5.9 mg, 0.109 mmol), and TCEP (23.9 mg, 0.0833 mmol) in 1.6 mL of DMF, and the reaction mixtures were incubated at room temperature overnight. This procedure allows Fmoc-protected 2-aminoethanethiols immobilized on MNPs through Michael addition with all available maleimide moieties on the surface. TCEP was used for in situ generation of free sulfhydryl group. The Reaction mixture was washed with DMF for 5 times. The available maleimide moiety was quantified through Fmoc quantification of MNP@SiO₂(FITC)-PEG/NH-maleimide-S—NH-Fmoc particle using same procedure.

General sequence-independent procedure to create sulfhydryl group on the antibody for specific immobilization on nanoparticles: CD-10 antibody solution (50 μL, MW: 100 kDa, 200 μg·mL⁻¹ in PBS) was pretreated with 10 μL of 0.5 M EDTA solution, followed by addition of 2-mercaptoethylamine solution (5 μL, 0.779 mmol) in 500 μL PBS solution containing 10 μL of 0.5 M EDTA solution. After incubation at 37° C. for 90 min, reduced half-antibody fragments were purified by gel filtration using Sephadex G-25 and detected by Bradford assay using manufacturer's protocol. Combined half-antibody fragments were immediately incubated with MNP@SiO₂(FITC)-PEG/NH-maleimide (0.8 mL, maleimide moieties on MNP=3.6 μmol·g⁻¹, 22.9 mg·mL⁻¹ in PBS) at 37° C. overnight. Antibody-immobilized MNP, MNP@SiO₂(FITC)-Ab_{CD-10}, was precipitated by centrifugation at 13,000 rpm for 20 min and the supernatant was removed. PBS 1× solution (1 mL) was added and resuspended by voltexing for 6 hours.

Cell culture: SP2/0, A549, and MCF-7 were ordered from American Type Culture Collection (ATCC, Manassas, Va.). These cells were grown in Dulbecco's Modified Eagle Medium (DMEM, 2 mL, culture media; Cambrex Bio Science, Walkersville, Md.) containing with 10% fetal bovine serum (FBS v/v), 0.5% gentamicin (v/v), and nanoparticle solution (0.2 mg·mL⁻¹; 40 μl). All cells were cultured in Lab-Tek glass chamber slide (Nalge Nunc International, Naperville, Ind.) in order to observe fluorescence emission by confocal laser scanning microscopy (CLSM).

Protocol of Transmission Electron Microscope: To study the metabolism of nanoparticles, treatment cells were fixed with 1% glutaraldehyde and 1.5% paraformaldehyde in 0.1 M phosphate buffer, pH 7.2 at 4° C. The samples were sequentially washed with PBS followed by 0.1 M cacodylate buffer, pH 7.2 and post-fixed with 1% osmium tetroxide in 0.1 M cacodylate buffer for 1.5 hour at room temperature. The samples were then washed briefly with dH₂O and dehydrated through a graded ethanol series and infiltrated by using of propylene oxide and EPON epoxy resin (EmBed 812, Electron Microscopy Sciences), and finally embedded with only epoxy resin. The epoxy resin mixed samples were loaded into capsules and polymerized at 60° C. for 24 hours. Thin sections were made using a RMC MT-X ultramicrotome and collected copper grids and not stained any reagent for detecting of nanoparticles into the cells. Images were collected using a JEOL (JEM-1011) transmission electron microscope at 80 kV with a GATAN digital camera.

Standard Fmoc Quantification Protocols: Fmoc protected MNP@SiO₂(OD)-PEG/NH₂s were precisely weighed about 2 mg in an Eppendorf tube through the separation by centrifugation followed by drying under high vacuum overnight. Precipitated MNPs were resuspended with 0.8 mL of DMF by sonication for 20 minute, followed by addition of 0.2 mL of piperidine for Fmoc cleavage. The cleavage cocktail with MNPs was sonicated for 20 minutes. The supernatant was collected by centrifugation at 13,000 rpm for 20 minutes, and the UV absorbance of Fmoc solution (0.9 mL in cell) was obtained at 300 nm. 7800 is the extinction coefficient (units mol⁻¹dm³cm⁻¹) at 300 nm. For greater accuracy, each sample was tested in heptaplicate and UV absorbance should lie between about 0.3 and 1.2 absorbance units.

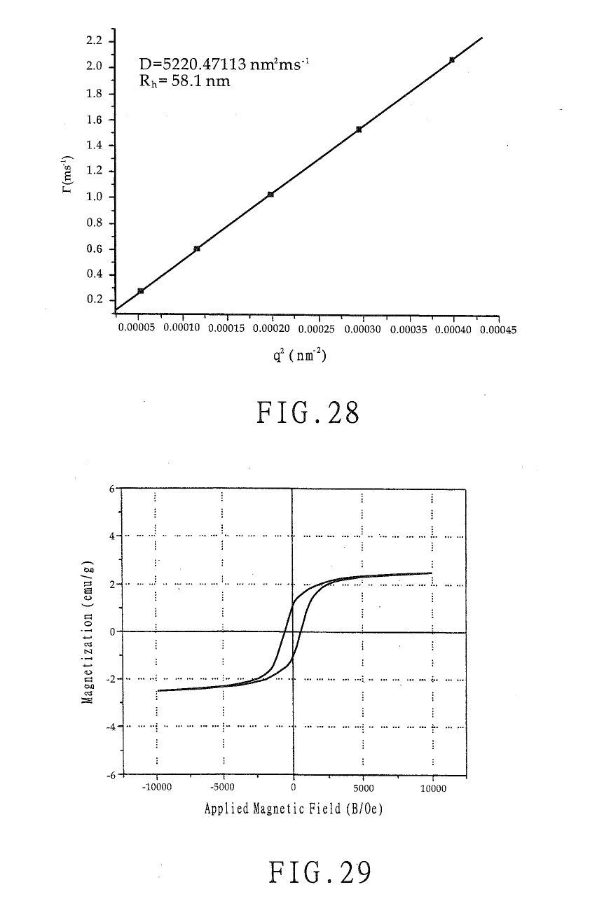

FIG. 28: X-ray powder diffractogram shows the typical diffraction pattern of spinel structure, confirming the core structure of magnetic Co-ferrite nanoparticle. The data was collected using Philips PW 3710 powder diffractometer equipped with Ni-filtered Cu K-radiation (λ=1.5406 Å). Then, according to the Scherrer equation, the roughly spherical particles size is related to the width of the diffracted beams. Using the full width at half-maximum of the most intense X-ray peak, which corresponds to the [311] one, it gives in the case of our samples the nanoparticles mean diameter of 9 nm. Co ferrite Cubic cells deduced from the analysis of x-ray diffraction patterns (lattice parameter, aₑₓₚ=0.835). Increasing of peak broadness at the low angle was occurred from amorphous silica shell.

FIG. 29. Dynamic light scattering measurements were performed using a UNIPHASE He—Ne laser operating at 632.8 nm. The maximum operating power of the laser was 30 mW. The detector optics employed optical fibers coupled to an ALV/SO-SIPD/DUAL detection unit, which employed an EMI PM-28B power supply and ALV/PM-PD preamplifier/discriminator. The signal analyzer was an ALV-5000/E/WIN multiple tau digital correlator with 288 exponentially spaced channels. Its minimum real sampling time is 10⁻⁶s and a maximum of about 100 s. A lens with a focal length of 200 mm narrowed the incident beam to reduce the thermal lensing effect and to increase the coherence area. The scattered beam passed through two pin holes before reaching the PMT. A scattering cell (10 mm diameter cylindrical) was placed in a temperature controlled bath of index matching liquid, decaline. All of the experiments were performed at 25±0.1° C.

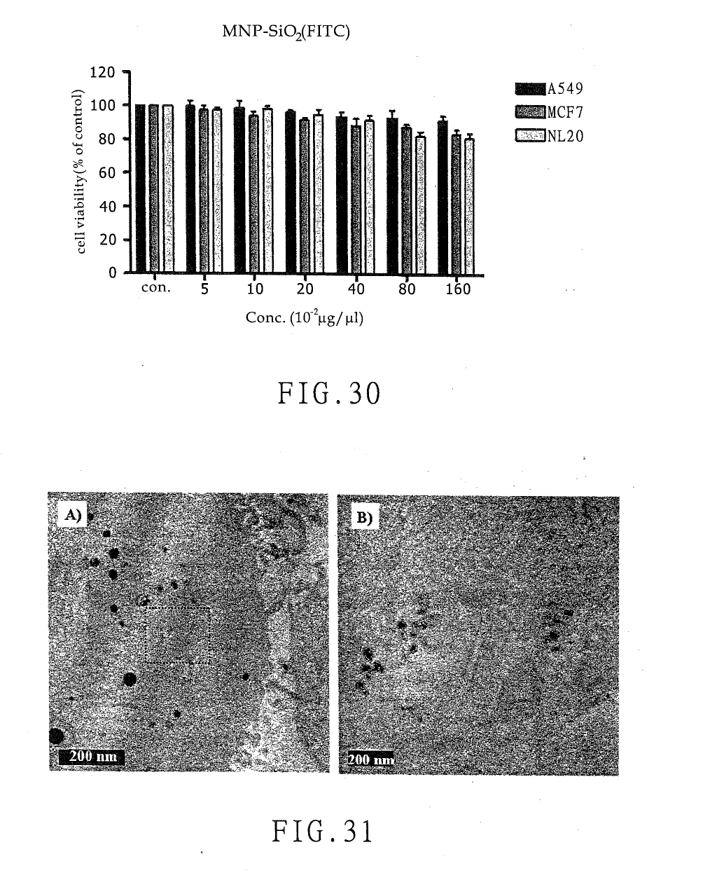

FIG. 30. Magnetic properties of core-shell nanoparticles were measured by Vibrating Sample Magnetometer (VSM, Lake Shore Model 7304). The nanoparticles were already described in, for example, FIG. 21. The coercivity (HO value was exactly same for bare Co ferrite nanoparticles.

FIG. 31. The cells were incubated in a 96-well plate. At the end of the incubation period, 3(4,5-dimethylthiazol-2-yl) 2,5-diphenyltetrazolium bromide (MTT; Sigma-Aldrich) in PBS (0.2 mg mL⁻¹) was added to each well (final concentration of 0.4 mg mL⁻¹) and cultures were incubated in 5% CO₂ for 4 h at 37° C. Then the culture medium was carefully removed by pipetting and formazan crystals generated by dehydronase activity in mitochondria, which only occurs in living cells, were dissolved in DMSO for the analysis. After 10 min agitation on a shaker, absorbance was measured at 490 nm and 620 nm for test and reference solutions, respectively.

### PART THREE

### References

33. a) D. Weller, M. F. Doerner, Annu. Rev. Mater. Sci. 2000, 30, 611-644; b) H. Zeng, J. Li, J. P. Liu, Z. L. Wang, S. H. Sun, Nature 2002, 420, 395-398; c) C. Ross, Annu. Rev. Mater. Sci. 2001, 31, 203-235; d) S. D. Jacobs, W. Kordonski, I. V. Prokhorov, D. Golini, G. R. Gorodkin, T. D. Strafford, U.S. Pat. No. 5,795,212, 1998.
34. a) I. Willner, E. Katz, Angew. Chem. Int. Ed. 2003, 42, 4576-4588; b) K. A. Hinds, J. M. Hill, E. M. Shapiro, M. O. Laukkanen, A. C. Silva, C. A. Combs, T. R. Varney, R. S. Balaban, A. P. Koretsky, C. E. Dunbar, Blood 2003, 102, 867-872; c) H. L. Grossman, W. R. Myers, V. J. Vreeland, R. Bruehl, M. D. Alper, C. R. Bertozzi, J. Clarke, Proc. Natl. Acad. Sci. USA 2004, 101, 129-134; d) P. S. Doyle, J. Bibette, A. Bancaud, J. L. Viovy, Science 2002, 295, 2237-2237.
35. a) Q. A. Paukhurst, J. Connolly, S. K. Jones, J. Dobson, J. Phys. D: Appl. Phys. 2003, 36, R167-R181; b) P. Tartaj, M. P. Morales, S. Veintemillas-Verdaguer, T. González-Carre\check{n}o, C. J. Sema, J. Phys. D: Appl. Phys. 2003, 36, R182-R197; c) C. C. Berry, A. S. G. Curtis, J. Phys. D: Appl. Phys. 2003, 36, R198-206.
36. a) H. H. Bengele, S. Palmacci, Magn. Reson. Imaging 1994, 12, 433-442; b) L. Josephson, J. Lewis, P. Jacobs, P. F. Hanh, D. D. Stark, Magn. Reson. Imaging 1988, 6, 647-653; c) M. I. Papisov, A. Bogdanov, B. Schaffer, N. Nossiff, T. Shen, R. Weissleder, T. J. Brady, J. Magn. Magn. Mater. 1993, 122, 383-386.
37. a) O. Veiseh, C. Sun, J. Gunn, N. Kohler, P. Gabikian, D. Lee, N. Bhattarai, R. Ellenbogen, R. Sze, A. Hallahan, J. Olson, M. Zhang, NanoLett. 2005, 5, 1003-1008; b) Y. Jun, Y. Huh, J. Choi, J. Lee, H. Song, S. Kim, S. Yoon, K. Kim, J. Shin, J. Suh, J. Cheon, J. Am. Chem. Soc. 2005, 127, ASAP; c) J. Lange, R. Kotitz, A. Haller, L. Trahms, W. Semmler, W. Weitschies, J. Magn. Magn. Mater. 2002, 252, 381-383; d) F. Patolsky, Y. Weizmann, E. Katz, I. Willner, Angew. Chem. 2003, 115, 2474-2478.
38. a) C. Kirchner, T. Liedl, S. Kudera, T. Pellegrino, A. M. Javier, H. E. Gaub, S. Stölzle, N. Fertig, W. J. Parak, Nano Lett. 2005, 5, ASAP; b) D. A. Limaye, Z. A. Shaikh, Toxicol. Appl. Pharmacol. 1999, 154, 59-66; c) R. A. Goyer, Am. J. Clin. Nutr. 1995, 61, 646s-650s.
39. a) M. A. Correa-Duarte, M. Giersig, N. A. Kotov, L. M. Liz-Marzán, Langmuir 1998, 14, 6430-6435; b) L. M. Rossi, L. Shi, F. H. Quina, Z. Rosenzweig, Langmuir 2005, 21, 4277-4280; c) Z. F. Li, E. Ruckenstein, Nano Lett. 2004, 4, 1463-1467; d) S. Santra, H. Yang, D. Dutta, J. T. Stanley, P. H. Holloway, W. Tan, B. M. Moudgil, R. A. Mericle, Chem. Commun. 2004, 2810-2811.
40. a) C. Graf, D. L. J. Vossen, A. Imhof, A. van Blaaderen, Langmuir 2003, 19, 6693-6700; b) C. R. Vestal, Z. J. Zhang, Nano Lett. 2003, 3, 1739-1743; c) T. Tago, T. Hatsuta, K. Miyajima, M. Kishida, S. Tashiro, K. Wakabayashi, J. Am. Ceram. Soc 2002, 85, 2188-2194; d) F. Grasset, N. Labhsetwar, D. Li, D. C. Park, N. Saito, H. Haneda, O. Cador, T. Roisnel, S. Mornet, E. Duguet, J. Portier, J. Etourneau, Langmuir 2002, 18, 8209-8216.
41. a) C. Barbé, J. Bartlett, L. Kong, K. Finnie, H. Q. Lin, M. Larkin, S. Calleja, A. Bush, G. Calleja, Adv. Mater. 2004, 16, 1959-1966; b) T. Nann, P. Mulvaney, Angew. Chem. Int. Ed. 2004, 43, 5393-5396.
42. a) Y. Lu, Y. Yin, B. T. Mayers, Y. Xia, Nano Lett. 2002, 2, 183-186; b) T. J. Yoon, J. S. Kim, B. G. Kim, K. N. Yu, M. H. Cho, J. K. Lee, Angew. Chem. Int. Ed. 2005, 44, 1068-1071.
43. a) H. Ow, D. R. Larson, M. Srivastava, B. A. Baird, W. W. Webb, U. Wiesner, Nano Lett. 2005, 5, 113-117; b) C. Graf, W. Schärtl, K. Fischer, N. Hungenberg, M. Schmidt, Langmuir 1999, 15, 6170-6180.
44. a) H. Gu, P. Ho, K. W. T. Tsang, L. Wang, B. Xu, J. Am. Chem. Soc. 2003, 125, 15702-15703; b) D. Wang, J. He, N Rosenzweig, Z. Rosenzweig, Nano Lett. 2004, 4, 409-413; c) J. Nam, C. S. Thaxton, C. A. Mirkin, Science 2003, 301, 1884-1886; d) B. Zebli, A. S. Susha, G. B. Sukhorukov, A. L. Rogach, W. J. Parak, Langmuir 2005, 21, 4262-4265.
45. a) J. M. Saul, A. Annapragada, J. V. Natarajan, R. V. Bellamkonda, J. Control. Release 2003, 92, 49-67; b) X. Gao, Y. Cui, R. M. Levenson, L. W. K. Chung, S, Nie, Nat. Biotechnol. 2004, 22, 969-976.
46. a) S. Kim, S. Kim J B. Tracy, A. Jasanoff, M G. Bawendi, J. Am. Chem. Soc. 2005, 127, 4556-4557; b) Z. Li, L. Wei, M. Gao, H. Lei. Adv. Mater. 2005, 17, 1001-1005; c) M. Zhao, M. F. Kircher, L. Josephson, R. Weissleder, Bioconjugate Chem. 2002, 13, 840-844.
47. a) D. P. Tang, R. Yuan, Y. Q. Chai, X. Zhong, Y. Lin, J. Y. Dai, L. Y. Zhang, Anal. Biochem. 2004, 333, 345-350; b) S. Balthasar, K. Michaelis, N. Dinauer, H. von Briesen, J. Kreuter, K. Langer, Biomaterials 2005, 26, 2723-2732.
48. a) S. Santra, P. Zhang, K. Wang, R. Tapec, W. Tan, Anal. Chem. 2001, 73, 4988-4993; b) A. Vora, L. Frost, A. Goodeve, G. Wilson, R. M. Ireland, J. Lilleyman, T. Eden, I. Peake, S. Richards, Blood 1998, 92, 2334-2337.
49. a) X. Wu, H. Liu, J. Liu, K. N. Haley, J. A. Treadway, J. P. Larson, N. Ge, F. Peale, M. P. Bruchez, Nat. Biotechnol. 2003, 21, 41-46; b) D. Artemov, N. Mori, R. Ravi, Z. M. Bhujwalla, Cancer Res. 2003, 63, 2723-2727.
50. a) D. Pantarotto, J. P. Briand, M. Prato, A. Bianco, Chem. Commun. 2004, 16-17; b) Q. Lu, J. M. Moore, G. Huang, A. S. Mount, A. M. Rao, L. L. Larcom, P. C. Ke, Nano Lett. 2004, 4, 2473-2477; c) A. Bianco, J. Hoebeke, S. Godefroy, O. Chaloin, D. Pantarotto, J. P. Briand, S. Muller, M. Prato, C. D. Partidos, J. Am. Chem. Soc. 2005, 127, 58-59.
51. a) N. A. M. Verhaegh, A. Van Blaaderen, Langmuir 1994, 10, 1427-1438; b) A. Van Blaaderen, V. Vrij, Langmuir 1992, 8, 2921-2931; c) R. P. Bagwe, C. Yang, L. R. Hilliard, W. Tan, Langmuir 2004, 20, 8336-8342.

No admission is made that any references cited herein are prior art.

Although the various embodiments have been described herein relative to specific materials, examples, and embodiments, it is not so limited. There are numerous variations and modifications that will be readily apparent to those skilled in the art to which it pertains in the light of the above teachings. One skilled in the art can recognize that other method for practicing these embodiments can be carried out not expressly and specifically described herein.

What is claimed is:

1. A composition comprising:
a plurality of particles having an average particle size of about 100 nm or less,
wherein the particles comprise:
a core comprising magnetic material, and
a glassy inorganic oxide shell disposed around the core which is covalently bound to at least one luminescent organic dye which is distributed through the glassy inorganic oxide shell, wherein the shell further comprises a surface agent which is covalently bound to the shell and enhances the particle cellular uptake.

2. The composition according to claim 1, wherein the nanoparticles are free of components which provide a specific recognition.

3. The composition according to claim 1, wherein the core comprises a particle comprising an organic polymer stabilizer.

4. The composition according to claim 1, wherein the particles do not photobleach.

5. The composition according to claim 1, wherein the shell is substantially free of surface ionic groups.

6. The composition according to claim 1, wherein the shell comprises ionic surface groups or nucleophilic groups.

7. The composition according to claim 1, wherein the particles are dispersed in water or polar solvent.

8. The composition according to claim 1, wherein the particles are surface functionalized with an agent for specific binding.

9. The composition according to claim 1, wherein the particles are surface hybridized with a nucleic acid agent.

10. The composition according to claim 1, wherein the particles have an average particle size of about 30 nm to about 80 nm, wherein the core comprises iron and a PVP stabilizer, wherein the shell comprises silica, wherein the surface agent comprises ethylene oxide repeat units, and wherein the particles do not photobleach.
